(19) United States
(12) Patent Application Publication
(10) Pub. No.: US 2024/0335477 A1
(43) Pub. Date: Oct. 10, 2024

(54) CELL POPULATIONS IN THE ANORECTAL TRANSITION ZONE WITH TISSUE REGENERATIVE CAPACITY, AND METHODS FOR ISOLATION AND USE THEREOF

(71) Applicant: Curileum Discovery Ltd, Harrow (GB)

(72) Inventors: Priti KALRA, London (GB); Jeffrey G. MOORE, London (GB)

(73) Assignee: Curileum Discovery Ltd, Harrow (GB)

(21) Appl. No.: 18/682,302

(22) PCT Filed: Aug. 12, 2022
(86) PCT No.: PCT/IB2022/000456

§ 371 Date: Feb. 8, 2024

Related U.S. Application Data
(60) Provisional application No. 63/260,226, filed on Aug. 12, 2021.

(51) Int. Cl. A61K 35/37; A61L 27/24; A61L 27/36; A61L 27/38; A61P 1/00; C12N 5/071

(52) U.S. Cl. CPC A61K 35/37; A61L 27/24; A61L 27/3666; A61L 27/3834; A61P 1/00; C12N 5/068

(57) ABSTRACT

Provided herein are compositions of anorectal transition zone stem cells, including multipotent and progenitor cells, to treat anal fistulas, for example, refractory complex perianal fistulas associated with Crohn's disease or of unknown aetiology. Also provided are pharmaceutical compositions of the anorectal transition zone stem cells, including multipotent and progenitor cells, in a pharmaceutically acceptable carrier.

### BACKGROUND

Transition zones of the gastrointestinal tissues exist between two different epithelial tissue types. Anorectal transition tissue is at the junction of rectal columnar epithelium of endodermal embryological origin and anal stratified squamous, non-keratinised epithelium of ectodermal embryological origin (McNairn and Guasch (2011), “Epithelial transition zones: merging microenvironments, niches, and cellular transformation,” Eur J Dermatol, 21(Suppl 2): 21-8). Histopathological labelling studies of mouse and human anorectal transition zone tissue has identified cells expressing p63, a marker for basal cells; cytokeratin 7 (CK7), a marker for simple columnar cells (Yang et al. (2015), “Microanatomy of the cervical and anorectal squamocolumnar junctions: a proposed model for anatomical differences in HPV related cancer risk,” Mod Pathol, 28(7): 994-1000); and CD34, a marker for stem cells and progenitor cells (McNairn, supra). Further studies in mice have supported the presence of stem cells and progenitor cells in the anorectal transition zone using a label retaining cell assay and immunohistochemistry confirming expression of p63, CK7, CD34 and identifying expression of SOX2, a pluripotent stem cell marker (Runck et al. (2010), “Identification of epithelial label-retaining cells at the transition between the anal canal and the rectum in mice,” Cell Cycle, 9(15): 3039-45).

Nearly 85% of fistulas in Crohn's patients originate at the anorectal transition zone, also called the dentate line (Sheikh (2012), “Controversies in fistula in ano,” Indian J. Surg., 74(3): 217-220). Perianal fistulising disease is an incurable and debilitating condition in Crohn's disease that affects up to 40% of patients. Perianal fistulas of unknown actiology and Crohn's patients have a frequency of 1:10,000 in the general population (Eglinton et al. (2012), “The spectrum of perianal Crohn's disease in a population-based cohort,” Dis. Colon Rectum, 55(7): 773-777). A tunnel initiates at the juncture of the rectum and the anal canal to form a fistula (Eglinton, supra). The fistula tunnel passes through muscle, fat, and opens outside the body at the buttock skin. Exposure of the fistula to gut contents elicits a state of chronic inflammation that requires periodic clearing of tissue debris, called granulation tissue.

No treatments durably heal a cleaned fistula. Volume-retaining scaffolding products to infill the tract, such as fibrin glue and porcine plugs, are prone to eliciting abscess formation (Buchanan et al. (2003), “Efficacy of fibrin sealant in the management of complex anal fistula: a prospective trial,” Dis Colon Rectum, 46(9): 1167-1174) and spontaneous expulsion (Amrani et al. (2008), “The Surgisis AFP anal fistula plug: a new and reasonable alternative for the treatment of anal fistula,” Gastroenterol Clin Biol, 32(11): 946-948), respectively.

Current treatments reduce symptoms that extend the period between fistula cleanings. Antibiotics, immunosuppressants, and TNF-alpha inhibitors provide limited relief, while having side effects and unsatisfactory therapeutic outcomes (Kumar and Thompson (2013), “Endoscopic therapy for postoperative leaks and fistulae,” Gastrointest. Endosc. Clin. N. Am., 23(1): 123-136; Swaminath et al. (2014), “Use of methotrexate in inflammatory bowel disease in 2014: A User's Guide.” World J. Gastrointest. Pharmacol. Ther, 5(3): 113-121). Injecting allogeneic adipose-derived mesenchymal stem cells (AdMSC or ASC, Alofisel™, darvadstrocel) directly in tissue along the fistula tract has shown an additional benefit of 14% over the placebo control in a single comparative clinical trial, with no statistically significant effect after one year of follow-up (NICE Technology appraisal guidance [TA556]).

### SUMMARY

The invention is based, in part, upon the discovery of compositions for use in treating a fistula in a subject in need thereof, methods of making compositions useful in the treatment of fistulas, and methods of treating a fistula with such a composition.

In one aspect, provided herein are compositions for treating a fistula (e.g., an anal fistula), the composition comprising isolated anorectal transition zone (ATZ) cells and a pharmaceutically acceptable carrier. The ATZ cells comprise multipotent stem cells and/or progenitor cells. The cells can be allogeneic cells or autologous cells. In certain embodiments, the ATZ cells comprise ATZ stem cells that can differentiate into endodermal cells (e.g., that express SOX17), mesodermal cells (e.g., that express BRACHURY), ectodermal cells (e.g., that express PAX6 and/or NESTIN), or a combination thereof. The ATZ stem cells can be in vitro expanded ATZ (eATZ) stem cells.

In certain embodiments, the ATZ stem cells express one or more of CD34, CD117, and/or CD184, for example, as detected by flow cytometry (e.g., with antibodies or other ligands that bind to cell surface markers). Alternatively, or in addition, the ATZ stem cells do not express detectable levels of CD45, for example, as detected by flow cytometry. Furthermore, in certain embodiments, the ATZ stem cells express NANOG and/or OCT4A.

The ATZ cells can be combined with an exogenous biocompatible scaffold, for example, a synthetic scaffold or a biological scaffold. Depending upon the circumstances, the scaffold can comprise a collagen-based scaffold.

The ATZ cells can be porcine cells or human cells.

In certain embodiments, the composition is cryopreserved. To facilitate the cryopreservation, the composition may comprise a suitable cryopreservation media.

In another aspect, the invention provides pharmaceutical dosage forms comprising the compositions disclosed herein, optionally wherein the dosage form is disposed within a container or capsule.

In another aspect, the invention provides methods of preparing the pharmaceutical compositions (e.g., any of the compositions as disclosed herein). The methods comprise (a) harvesting ATZ tissue from a donor (e.g., a porcine donor or a human donor); (b) enzymatically digesting the tissue to prepare a cell suspension; (c) optionally combining at least a portion of the cell suspension with a cryopreservation media and cryopreserving the cell suspension; and (d) combining the cell suspension of step (b) or optional step (c) with a pharmaceutically acceptable carrier.

The cell suspension comprises ATZ cells, which can include multipotent stem cells and/or progenitor cells. In certain embodiments, the ATZ stem cells can differentiate into endodermal cells (e.g., that express SOX17), mesodermal cells (e.g., that express BRACHURY), ectodermal cells (e.g., that express PAX6 and/or NESTIN), or a combination thereof.

Depending upon the circumstances, the ATZ stem cells express one or more of CD34, CD117, and CD184, for example, as detected by flow cytometry. Alternatively, or in addition, the ATZ cells do not express detectable levels of CD45, for example, as detected by flow cytometry. Furthermore, the ATZ stem cells can express NANOG and/or OCT4A. The ATZ cells can be allogeneic or autologous cells.

In certain embodiments, the cells in the suspension (e.g., ATZ stem cells) are expanded in vitro.

Depending upon the circumstances, in step (d) of the method described above, cells in the cell suspension are combined with an exogenous biocompatible scaffold, for example, a synthetic scaffold or a biological scaffold. In certain embodiments, the scaffold comprises a collagen-based scaffold.

In another aspect, the invention provides pharmaceutical compositions produced by the methods disclosed herein.

In another aspect, the invention provides methods for treating a fistula (e.g., an anal fistula and/or a refractory fistula) in a subject in need thereof, the methods comprising administering into the fistula an effective amount of a pharmaceutical composition disclosed herein, or the dosage form, thereby to treat the fistula.

In some embodiments, the methods close the fistula, for example, with new fibrotic tissue. In some embodiments, the subject has Crohn's disease or a fistula of unknown aetiology.

### BRIEF DESCRIPTION OF THE DRAWINGS

FIG. 1 shows isolation of anorectal transition zone crypts from porcine tissue: (a) shows resected colorectum tissue; (b) shows exposed mucosal epithelium, (c) shows excised dentate line, (d) shows freshly isolated ATZ crypts, (e) shows freshly isolated ATZ crypts, (f) shows freshly isolated ATZ crypts, (g) shows freshly isolated bifurcating ATZ crypt, (h) shows freshly isolated ATZ submucosal gland, and (i) shows freshly isolated ATZ submucosal gland

FIG. 2 shows isolation of anorectal transition zone crypts from Crohn's disease patient tissue: (a), shows resected anorectum tissue, (b) shows excised mucosal epithelium, (c) shows brightfield images of freshly isolated ATZ crypts, and (d) shows brightfield images of freshly isolated ATZ crypts.

FIG. 3 shows (a) crypt organoid development in porcine GI tissue, and (b) crypt organoid development in Crohn's disease patient rectum and ATZ tissue.

FIG. 4 shows increased progenitor capacity of single cell preparations of porcine ATZ crypts as compared to rectal crypts. FIG. 4(a) shows porcine ATZ and rectal crypt growth over 4 weeks. FIG. 4(b) shows increased plating efficiencies over two months of crypt organoids derived from ATZ cells compared to rectal cells. Cells were isolated and replated after 1 month.

FIG. 5 shows protein expression of stem cell and progenitor cell markers on freshly isolated crypts and crypt organoids from pigs and a Crohn's disease patient. FIG. 5(a) shows CD117 expression on crypt-derived single cells from porcine GI tissues. FIG. 5(b) shows CD34 expression on porcine fresh crypt-derived single cells from porcine GI tissues. FIG. 5(c) shows double labelling by flow cytometry of CD34 and CD117 expression on fresh porcine ATZ crypt cells. FIG. 5(d) shows stem cell and progenitor marker expression on porcine crypt organoids. SI is shown on the left bar, rectum is shown in the middle bar, ATZ is shown on the right bar. FIG. 5(e) shows the indicated developmental lineage marker expression on fresh porcine ATZ crypt-derived cells. FIG. 5(f) shows stem cell and progenitor marker expression on Crohn's disease patient rectal crypt-derived organoids.

FIG. 6 shows mRNA profiling from freshly isolated crypts and crypt organoids of the porcine anorectal transition zone. FIG. 6(a) mRNA profiling of freshly isolated porcine ATZ crypt cells. FIG. 6(b) shows mRNA profiling of organoids derived from porcine ATZ crypt cells.

FIG. 7 shows flow cytometric analysis of stem cell and progenitor cell markers to assess growth media at day 14 to promote porcine ATZ crypt stem cell expansion, wherein FIG. 7(a) shows CD117 expression, FIG. 7(b) shows Brachyury expression, and FIG. 7(c) shows SOX17 expression.

FIG. 8 shows that porcine ATZ crypt cells generate mature cells of the endoderm line. FIG. 8(a)-(d) shows brightfield images of organoid development from days 1, 4, 7, and 12, respectively. FIG. 8(e)-(h) shows DAPI staining of nuclei and immunocytochemistry images of organoids. FIG. 8(e) shows lysosome expression, FIG. 8(f) shows Ki67 expression, FIG. 8(g) shows Muc-2 expression, FIG. 8(h) shows CK18 expression.

FIG. 9 shows flow cytometry analysis of endodermal markers on ATZ organoids at days 0, 7, and 14.

FIG. 10 shows that porcine ATZ crypt cells generate mature cells of the mesoderm lineage. FIG. 10(a)-(d) shows brightfield images of blood vessel-like development, wherein FIG. 10(a) shows undifferentiated ATZ single cells at day 0, FIG. 10(b) shows ATZ single cells starting to form clusters at day 3, FIG. 10(c) shows blood vessel-like structures starting to form at day 8, FIG. 10(d) shows a network of blood vessel-like structures forming at day 10. FIG. 10(e) shows DAPI stained nucleus and immunocytochemistry of CD31 expressing endothelial-like cells, and FIG. 10(f) shows DAPI stained nucleus and immunocytochemistry of CD31 expressing blood vessel-like cells.

FIG. 11 shows that porcine ATZ crypt cells generate mature cells of the mesoderm lineage. FIG. 11 shows flow cytometric analysis of mesodermal markers, Brachyury and CD31, on ATZ organoids at days 0, 7, and 14.

FIG. 12 shows porcine ATZ crypt cells generate mature cells of the ectoderm lineage. FIG. 12(a) shows brightfield images of keratinocyte development from anal skin and ATZ crypts grown in KFSM. FIG. 12(b) shows immunocytochemistry of K14 and K15 markers on anal skin cultures. FIG. 12(c) shows immunocytochemistry of K14 and K15 markers on ATZ crypt cultures. FIG. 12(d) shows flow cytometry analysis of ectodermal markers PAX6, NESTIN, and CK14 on ATZ cultured cells on days 0, 7 and 14.

FIG. 13 shows brightfield images of porcine small intestine (SI) crypt single cells in medium to promote differentiation in the 3 developmental lineages at days 0, 7, and 14, wherein FIG. 13(a) shows culture in endoderm medium (OGM), FIG. 13(b) shows culture in mesodermal medium (MethoCult), FIG. 13(c) shows culture in ectodermal medium (KFSM).

FIG. 14 shows the results of an in vitro embryoid body assay to test pluripotency of porcine ATZ crypt single cells. FIG. 14(a) shows brightfield images of ATZ embryoid body development. FIG. 14(b) shows alkaline phosphatase staining of ATZ embryoid bodies. FIG. 14(c) shows immunostaining of ATZ embryoid bodies for expression of pluripotent stem cell markers SSEA4 and OCT4. FIG. 14(d) shows immunostaining of ATZ embryoid bodies with pluripotent stem cell markers SOX2 and TRA-1-60 expression. DAPI was used for nucleus staining.

FIG. 15 shows ATZ crypt organoids in vitro biocompatibility with a synthetic scaffold control (PeptiGel or “PG”). FIG. 15(a) shows brightfield images of ATZ organoids grown in 2%, 5%, and 10% PG or Matrigel® Matrix scaffold control and cultured up to 19 days, FIG. 15(b) shows the number of ATZ organoids cultured in PeptiGel or Matrigel® Matrix in culture up to 24 days.

FIG. 16 shows photomicrographs of ATZ organoids interaction with PeptiGels, wherein FIG. 16(a) shows Alpha 4 RGD, FIG. 16(b) shows Alpha 4 IKVAV, FIG. 16(c) shows Alpha 4 YIGSR, FIG. 16(d) shows Alpha 4 GFOGER, FIG. 16(e) shows Alpha 4 IKVAV+YIGSR, FIG. 16(f) shows Matrigel.

FIG. 17 shows a schematic diagram of porcine perianal fistula creation and treatment.

FIG. 18(a)-(d) shows perianal fistula creation for the study in a porcine model.

FIG. 19 shows histological assessment with H&E staining of transections of porcine fistula tracts at day 90 after treatments, of fistula tracts, where FIG. 19(a) shows the no treatment control, FIG. 19(b) shows hydrogel scaffold control (PeptiGel), and FIG. 19(c) shows ATZ cells+PeptiGel scaffold.

FIG. 20 shows examples of histological assessment of porcine fistulas at day 90 after treatment. FIG. 20(a) shows ATZ cells+PeptiGel treatment: α-smooth muscle actin immunohistochemistry detecting blood vessels. FIG. 20(b) shows ATZ cells+PeptiGel treatment. Picrosirius red staining was used for collagen (visualised under polarised light).

FIG. 21 shows three images (a)-(c) of the “No treatment control” fistula track in the porcine anal fistula model in Examples 13-14.

FIG. 22 shows shows three images (a)-(c) of the “No treatment control” fistula track in the porcine anal fistula model in Examples 13-14.

FIG. 23 shows shows three images (a)-(c) of the “Permacol scaffold” only treatment in the porcine anal fistula model in Examples 13-14.

FIG. 24 shows shows three images (a)-(c) of the “ATZ cells+Permacol scaffold” treatment in the porcine anal fistula model in Examples 13-14.

### DETAILED DESCRIPTION

### Definitions

As used herein, the following terms and phrases shall have the meanings set forth below. Unless defined otherwise, all technical and scientific terms used herein have the same meaning as commonly understood by one of ordinary skill in the art to which this disclosure belongs.

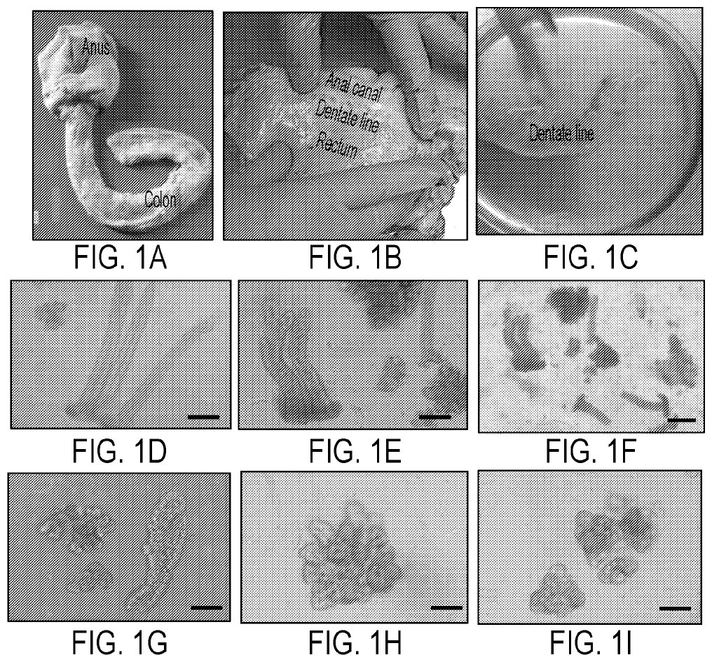

As used herein, the term “anorectal transition zone” or “ATZ” refers to the tissue interposed between the uninterrupted squamous epithelium of the anoderm and dentate (or pectinate) line below and the uninterrupted rectal columnar epithelium above. The dentate line is the junction between the superior and inferior anal canal. There are many differences between these two regions, including their embryological origins, innervation, venous and arterial supply, and lymphatic supply. Above the dentate line, the anal canal has an endodermal origin and is lined by simple columnar epithelia. Below the dentate line, the anal canal has an ectodermal origin and is predominantly lined by stratified squamous epithelium. The ATZ is typically 1-4 mm and can easily be identified and biopsied by those skilled in the art (see, e.g., FIG. 1).

As used herein, the term “anorectal transition zone cells” or “ATZ cells” refers to a mixed population of cells derived from anorectal transition zone tissue comprising crypts, submucosal glands, and other epithelial cells that include multipotent stem cells (i.e., cells that have the capacity to self-renew by dividing and to develop into multiple specialised cell types present in a specific tissue or organ), progenitor cells, and other cell types that originate from the anorectal transition zone tissue. Multipotent ATZ cells are stem cells that have the capacity to differentiate into multiple somatic cell lineages including endodermal cells (e.g., intestinal mucosa), mesodermal cells (e.g., blood vessels), and/or ectodermal cells (e.g., skin).

The term “crypts” refers to the crypts of Lieberkühn, structures below the surface of the intestinal mucosal lining, and comprising stem cells that are responsible for continuously regenerating intestinal mucosa throughout life.

As used herein, the term “stem cells” refers to undifferentiated or partially differentiated cells that can differentiate into various types of cells and proliferate indefinitely to produce more of the same stem cell (i.e., self-renewing).

As used herein, the term “progenitor cells” refers to stem cells with the potential to differentiate into a single cell type or lineage, and the term progenitor ATZ cells means progenitor cells derived from the ATZ.

As used herein, the term “multipotent cells” refers to stem cells with the potential to differentiate into at least two cell types or lineages, and the term multipotent ATZ cells means multipotent cells derived from the ATZ.

As used herein, the term “pluripotent cells” refers to stem cells with the potential to differentiate into each of the three primary groups of cells, i.e., ectoderm, mesoderm and endoderm, and the term pluripotent ATZ cells means multipotent cells derived from the ATZ.

As used herein, the terms “proliferation” and “proliferating” refer to an increase in cell number by mitosis.

The term “differentiation” refers to the formation of cells expressing markers known to be associated with cells that are more specialized and closer to becoming terminally differentiated cells incapable of further division or differentiation. For example, in a haematological context, differentiation can be seen in the production of functional cells of multiple cellular lineages (e.g., red blood cells, platelets, granulocytes, macrophages). The terms “further” or “greater” differentiation refers to cells that are more specialized and closer to becoming terminally differentiated cells incapable of further division or differentiation than the cells from which they were cultured. The term “final differentiation” refers to cells that have become terminally differentiated cells incapable of further division or differentiation.

The term “eATZ cells” refers to a cell suspension, for example, in aseptic buffered saline solution, containing expanded anorectal transition zone stem cells and progenitor cells for stem cell transplantation. The term “expanded” as used herein when referring to cells shall be taken to have its usual meaning in the art, namely cells that have been proliferated in vitro. eATZ cells can be expanded to provide a population of cells that retain at least one biological function of the non-expanded ATZ cells, typically the ability to form crypt organoids, under standard culture conditions. The expanded population of cells may retain the ability to differentiate into one or more cell types. In some embodiments, the eATZ cells retain at least one marker of ATZ stem cells selected from the group consisting of CD34, CD117, CD184, OCT4, NANOG, SOX17, BRACHURY, PAX6 and NESTIN.

The term “cellular composition” refers to a preparation of cells, which preparation may include, in addition to the cells, non-cellular components such as cell culture media, e.g., proteins, amino acids, nucleic acids, nucleotides, co-enzyme, antioxidants, metals and the like. Furthermore, the cellular composition can have components which do not affect the growth or viability of the cellular component, but which are used to provide the cells in a particular format, e.g., as polymeric matrix for encapsulation or a pharmaceutical preparation.

“Marker” refers to a biological molecule whose presence, concentration, activity, or phosphorylation state may be detected and used to identify the phenotype of a cell.

“Exogenous biocompatible scaffolds” are three-dimensional porous, fibrous or permeable volume-retaining biomaterials intended to permit transport of body liquids and gases, allowing for cellular interactions with minimum inflammation and toxicity. Optionally, such scaffolds are biodegradable. Examples of scaffolds can include a biological scaffold (e.g., a laminin or collagen-based scaffold) and synthetic scaffolds (e.g., non-biological polymers). Scaffolds may be in the physical form of a thread, sheet, paste, powder, or liquid. Scaffolds can be used in vitro and in vivo.

The term “fistula” refers to an abnormal passage or communication or connection, usually between two internal organs or leading from an internal organ to the surface of the body. Examples of fistulae include, but are not limited to, perianal fistula, anorectal fistula or fistula-in-ano, cervical fistula, enterovaginal fistula, and rectovaginal fistula. As used herein, the term “anal fistula” includes perianal fistula, anorectal fistula, and fistula-in-ano.

A single “tract” fistula, or simple fistula, has one internal opening and one external opening. A fistula with “multiple tracts” has more than one external opening and/or more than one internal opening. A multiple tract fistula often has different branches. Each external opening typically represents a tract.

A “complex perianal fistula” is a perianal fistula having one or more of: (i) high inter-, trans, extra- or supra-sphincteric origin; (ii) external openings; or (iii) associated collections. In some embodiments, the complex perianal fistula may have 2 or more internal openings and 3 or more external openings. In some embodiments, the complex perianal fistula may have been draining for at least 3, 4, 5, 6 weeks or more prior to treatment according to the disclosure.

The phrase “pharmaceutically acceptable” as used herein refers to those compounds, materials, compositions, and/or dosage forms which are, within the scope of sound medical judgment, suitable for use in contact with the tissues of human beings and animals without excessive or unacceptable toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

The term “phenotype” as used herein refers to the observable characteristics of a cell, such as size, morphology, RNA expression, protein expression, or other properties.

As used herein, the term “refractory” means resistant to standard treatments. In the case of the fistulas described herein, refractory fistulas can include fistulas that are not healed or closed by prior art treatments including, but not limited to, administration of antibiotics, immunosuppressants, TNF-alpha inhibitors, methotrexate, or introduction of volume-retaining scaffolding products (e.g., fibrin sealants), fistula plugs, adipose-derived mesenchymal stem cells (e.g., Alofisel™, darvadstrocel) directly into the fistula tract. In some embodiments, a refractory fistula may be successfully treated by the present invention to promote healing or closure of the fistula.

### Multipotent and Progenitor Atz Cells and Organoid Compositions

The multipotent and/or progenitor ATZ cells described herein can be isolated as single cell (or multiple cells) preparations derived from primary ATZ tissue. The ATZ cells described herein, including multipotent and progenitor ATZ cells, can be derived from intestinal crypts obtained by biopsy or can be derived from organoid structures differentiated ex vivo from ATZ cells. In some embodiments, the multipotent and progenitor ATZ cells are obtained from organoids derived from single primary ATZ cells or dissociated ATZ crypt cells.

For use in the methods of treatment described herein, the multipotent or progenitor ATZ cells are preferably autologous cells derived from a human patient or allogeneic cells derived from a human donor (preferably tissue-matched to reduce or avoid graft-versus-host or host-versus-graft immunoreactivity). Alternatively, the multipotent or progenitor ATZ cells can be derived from other mammalian species, and can be modified by methods known in the art to reduce or eliminate alloreactivity (e.g., gene editing to knock-out MHC Class I and/or Class II genes and/or the β2-microglobulin gene).

In some embodiments, the multipotent or progenitor ATZ cells can be in vitro expanded ATZ (eATZ) cells.

In some embodiments, the multipotent and progenitor ATZ cells, primary ATZ cells, crypts and/or organoids are combined in vivo or in vitro with an exogenous biocompatible scaffold, for example, a synthetic scaffold or a biological scaffold. In some embodiments, the scaffold comprises laminin and/or collagen (e.g., Permacol™ Paste, Medtronic PLC, Minneapolis, MN). In some embodiments, the scaffold is Matrigel® Matrix (Corning, New York) or a functional equivalent.

In some embodiments, the exogenous biocompatible scaffold is functionalised or is non-functionalised. The scaffold can be functionalised with peptides, for example, laminin (IKVAV and YIGSR), fibronectin (RGD), or collagen (GFOGER) peptides.

In some embodiments, the exogenous biocompatible scaffold comprises a mixture of functionalised and non-functionalised scaffolds components. In some embodiments, the functionalised scaffold components comprise between 0.1 to 10% of peptide. In some embodiments, the functionalised scaffold components comprise 0.1%, 0.5%, 1%, 2%, 3%, 4%, 5%, 6%, 7%, 8%, 9%, or 10% of peptide. In some embodiments, the exogenous biocompatible scaffold comprises between 99.9 to 90% non-functionalised scaffold. In some embodiments, the exogenous biocompatible scaffold comprises 99.9%, 99.5%, 99%, 98%, 97%, 96, 95%, 94%, 93%, 92%, 91%, or 90% non-functionalised scaffold, respectively. In some embodiments, the scaffold component is at least 0.1%, 0.5%, 1%, 5%, or 10%, 15%, 20%, 25%, 30%, 35%, 40%, 45%, 50%, 55%, 60%, 65%, 70%, 75%, 80%, 85%, 90%, 95%, or 100% functionalised scaffold. In some embodiments, the scaffold is at least 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, 98%, or 100% nonfunctionalised scaffold.

In some embodiments, the non-functionalised scaffold components comprises 98%, 95%, or 90% functionalised scaffold, respectively. In some embodiments, the scaffold component is at least 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 100% functionalised scaffold. In some embodiments, the scaffold is at least 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 100% nonfunctionalised scaffold.

The mixture of functionalised scaffolds can also comprise at least two functionalised scaffolds. The functionalised component of the final scaffold preparation can comprise any ratio of 1% to 99% of at least one functionalised scaffold and 99% to 1% of at least another functionalised scaffold. For instance, functionalised scaffold component comprises 50% IKVAV functionalised scaffold and 50% YIGSR functionalised scaffold. Alternatively, the mixture of the functionalised scaffold component can be 1% IKVAV and 99% YIGSR, or any ratio in between. For instance, the mixture of the functionalised scaffold can be 25% IKVAV and 75% YIGSR. Or in another instance, the mixture of the functionalised scaffold can be 75% IKVAV and 25% YIGSR. In some embodiments, the mixture of the functionalised scaffold comprises a collagen (e.g., GFOGER peptide) scaffold and another functionalised scaffold component. In some embodiments, the mixture of the functionalised scaffold is at least 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 100% IKVAV functionalised scaffold. In some embodiments, the mixture of the functionalised scaffold is at least 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 100% YIGSR functionalised scaffold. In some embodiments, the mixture of the functionalised scaffold is at least 0.1%, 0.5%. 1%, 5%, 10%, 15%, 20%, 25%, 30%, 35%, 40%, 45%, 50%, 55%, 60%, 65%, 70%, 75%, 80%, 85%, 90%, 95%, or 100% collagen functionalised scaffold.

In some embodiments, the scaffold comprises a functionalised collagen scaffold or a functional equivalent.

In some embodiments, the scaffold comprises a functionalised PeptiGel® Alpha 4 (Manchester BioGel) or a functional equivalent.

The exogenous biocompatible scaffold can also comprise cell culture medium components. In some embodiments, the scaffold comprises organoid growth medium (OGM), which can be purchased from commercial vendors (e.g., IntestiCult™, STEMCELL Technologies, Inc., Vancouver, Canada). In some embodiments, the ATZ cells or crypts can be cultured in OGM. In some embodiments, the ATZ cells or crypts can be cultured in a feeder-free medium that maintains human embryonic stem cells (ESCs) and/or induced pluripotent stem cells (iPSCs) in an undifferentiated state (e.g., mTesR™, STEMCELL Technologies, Inc., Vancouver, Canada). In some embodiments, the scaffold comprises such an ESC or iPSC feeder-free medium. In some embodiments, the ATZ cells or crypts cultured in OGM have a higher expression of mesoderm and endoderm lineage markers as compared to ATZ cells or crypts cultured in such an ESC or iPSC feeder-free medium. The matrix can also comprise keratinocyte serum-free medium (KSFM) for differentiation. Such KSFM medium is commercially available from vendors (e.g., Sigma-Aldrich, St. Louis, MO).

In another aspect, the disclosure provides for phenotypic characterisation of extracellular and intracellular protein and mRNA expression levels of stem cell and progenitor cell markers on freshly isolated single ATZ cells. ATZ cells can express at least one of CD34, CD117, and CD184 cell surface markers but do not express detectable levels of CD45 as detected by flow cytometry. In some embodiments, isolated porcine ATZ cells express OCT4 and NANOG.

ATZ stem cells can also express markers for stem cells of three developmental lineages: endoderm (SOX17), mesoderm (BRACHURY), and ectoderm (PAX6 and NESTIN). In addition, freshly isolated ATZ cells can express markers for pluripotent stem cells (NANOG and OCT4).

In some embodiments, the ATZ cells and/or crypts express genes associated with endoderm, ectoderm, and mesoderm lineages. In some embodiments, the ATZ cells and/or crypts express at least one gene marker selected from the group consisting of ALP, BRACHURY, BMP4, CD34, CD117 (KIT), CXCR4, CHGA, CK18, EPCAM, LGR5, LYS, MUC2, NANOG, PAX6, SOX17, and OCT4. In some embodiments, the ATZ cells and/or crypts express CD34. In some embodiments, the ATZ cells and/or crypts express CD117 (KIT) at a higher level as compared to small intestine, colon, and rectum crypt cells. In some embodiments, the ATZ cells and/or crypts express CD117 (KIT) at least 1.5-fold, 2-fold, 2.5-fold, 3-fold, 3.5-fold, 4-fold, 4.5-fold, 5-fold, 5.5-fold, 6-fold, 6.5-fold, 7-fold or higher as compared to small intestine, colon, and rectum crypt cells. In some embodiments, the ATZ cells and/or crypts express CD34. In some embodiments, the ATZ cells and/or crypts do not express detectable levels of CD45. In some embodiments, the ATZ cells and/or crypts express CD34 and do not express detectable levels of CD45.

In some embodiments, the ATZ crypts express at least one gene selected from the group consisting of ALP, BRACHURY, BMP4, CD34, CD117 (KIT), CXCR4, CHGA, CK18, EPCAM, LGR5, LYS, MUC2, PAX6, and SOX17. In some embodiments, the ATZ crypts express OCT4 or NANOG genes.

An aspect of the disclosure provides for phenotypic characterisation of extracellular and intracellular protein and mRNA expression levels of stem cell and progenitor cell markers on ATZ organoids in culture. In some embodiments, the ATZ organoids express genes associated with endoderm, ectoderm, and mesoderm lineages. In some embodiments, the ATZ organoids express at least one protein marker selected from the group consisting of ALP, BRACHURY, CD34, CD117 (KIT), CHGA, CK18, CXCR4, EPCAM, GD2, LGR5, LYS, MUC2, NESTIN, and PAX6. In some embodiments, the ATZ organoids express CD34. In some embodiments, the ATZ organoids do not express detectable levels of CD45. In some embodiments, the ATZ organoids express at least one gene selected from the group consisting of ALP, BRACHURY, BMP4, CD34, CD117 (KIT), CXCR4, CHGA, CK18, EPCAM, LGR5, LYS, MUC2, NANOG, PAX6, and SOX17.

In some embodiments, the ATZ organoids express at least one cell surface marker selected from the group consisting of CD31, CD34, CD117 (KIT), CXCR4 (CD184), CK14, CK15, CK18 GD2, SOX2, SSEA4, and TRA-1-60. In some embodiments, the ATZ organoids express CD34. In some embodiments, the ATZ organoids do not express detectable levels of CD45. In some embodiments, the ATZ organoids express CD34 but do not express detectable levels of CD45.

In some embodiments, the ATZ organoids express CD117 (KIT), CXCR4 (CD184), and/or GD2 at a higher level as compared to small intestine and rectum organoids. In some embodiments, the ATZ organoids express CD117 (KIT) at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids express CXCR4 (CD184) at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids express GD2 at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to small intestine and rectum organoids. In some embodiments, the ATZ organoids have reduced SOX17 expression as compared to ATZ crypt cells. In some embodiments, the ATZ organoids have reduced BRACHURY expression as compared to ATZ crypt cells. In some embodiments, the ATZ organoids have reduced CD31 expression as compared to ATZ crypt cells. In some embodiments, the ATZ organoids have reduced PAX6 expression as compared to ATZ crypt cells. In some embodiments, the ATZ organoids have increased LGR5 as compared to ATZ crypt cells. In some embodiments, the ATZ organoids have increased CD117 or CXCR4 as compared to ATZ crypt cells. In some embodiments, the ATZ organoids have increased K14 as compared to ATZ crypt cells. In some embodiments, the ATZ organoids have increased expression of CD117, CXCR4, LGR5, or K14 of at least 1.5-fold, 2-fold, 2.5-fold, 3-fold, 3.5-fold, 4-fold, 4.5-fold, 5-fold, 5.5-fold, 6-fold, 6.5-fold, or 7-fold higher as compared to ATZ crypt cells. In some embodiments, the ATZ organoids have decreased expression of SOX17, BRACHURY, CD31, or PAX6 of at least 1.5-fold, 2-fold, 2.5-fold, 3-fold, 3.5-fold, 4-fold, 4.5-fold, 5-fold, 5.5-fold, 6-fold, 6.5-fold, or 7-fold lower as compared to ATZ crypt cells.

Protein and nucleic acid expression can be determined by methods known to one of skill in the art. Such methods include, but are not limited to, polymerase chain reaction (PCR), RTPCR, q-RT-PCR, flow cytometry (combined with binding agents (e.g., labeled antibodies or ligands for cell surface markers), SDS-PAGE, mass spectrometry, immunoblotting (Western Blotting), immunofluorescence microscopy, fluorescence in situ hybridization, or any other technique known in the art.

In some embodiments, the ATZ organoids comprise proliferating cells, secretory cells, and cytokeratin. In some embodiments, the ATZ organoids can secrete LYSOZYME.

### Methods of Isolating and Producing Populations of Multipotent and Progenitor ATZ Cells

In another aspect, provided herein are methods of isolating and producing multipotent and/or progenitor ATZ cells. The ATZ cells can be produced by obtaining anorectal transition zone tissue from a mammalian subject (e.g., human, porcine) either from biopsied or resected tissue; washing tissues with a serum-free tissue culture medium optionally containing antibiotics and/or antimycotics; mincing the tissue; and enzymatically treating or digesting the tissues with a collagenase. Digestion can be stopped by, for example, adding protein such as albumin. Crypts can be isolated by rigorously stirring or shaking the tissue digest to release the crypts. Medium containing crypts can be passed through tissue strainers to remove large debris and centrifuged to sediment or pellet crypts; and the crypts can be resuspended in fresh medium to obtain a population of multipotent and/or progenitor ATZ cells.

Resuspended crypts can be enumerated under a microscope. Single cell preparations from crypts are generated by physical disruption with a needle/syringe and/or treatment with mild enzymatic cell dissociation reagents to obtain a population of multipotent and/or progenitor ATZ cells. These cells can then be embedded in an in vitro matrix scaffold (for example, as described above), and cultured with differentiation media to produce organoids.

In some embodiments, the enzymatic treatment of minced anorectal transition zone tissue releases intestinal crypts and submucosal glands. In some embodiments, the anorectal transition zone tissue is from a healthy human donor. In some embodiments, the anorectal transition zone tissue is from a diseased human donor.

The released intestinal crypts or multipotent and/or progenitor ATZ cells can then be differentiated into organoids after being placed in a growth medium or a scaffold (e.g., laminin, collagen, Permacol™, Matrigel® Matrix). Any suitable matrix and growth medium know in the art can be used. In some embodiments, the crypts are placed in a synthetic scaffold. In some embodiments, the scaffold is non-functionalised. In some embodiments, the scaffold is functionalised. Alternatively, the crypts can be further dissociated into single ATZ cells.

In some embodiments, the isolated ATZ-derived crypt cells have increased multipotent and/or progenitor capacity as compared to cells derived from rectum crypts.

In another aspect, the invention provides for in vitro growth and maintenance of ATZ stem cells (including multipotent and/or progenitor cells) by culturing crypts or single cells embedded in a scaffold (e.g., Permacol™, Matrigel® Matrix) with specialized intestinal organoid growth medium (e.g., IntestiCult™) that promotes perpetual growth and differentiation. OGM such as IntestiCult™, MethoCult™ (STEMCELL Technologies, Inc., Vancouver, Canada), or Keratinocyte Serum-Free Medium (e.g., Sigma-Aldrich, St. Louis, MO) are commercially available. ATZ organoid cultures are refreshed on periodic basis by breaking up organoids and replating in OGM.

In another aspect, the invention provides for methods of in vitro expansion of ATZ stem cells (including multipotent and/or progenitor cells) in preparation for treating fistulas. The methods can include repeated passaging of ATZ organoids. The methods can further include confirmation that harvested cells maintain phenotypic properties determined, for example, by protein and mRNA expression levels of cell surface and intracellular stem cell markers and replating efficiencies. Once the properties of ATZ cells are validated for a cell source, a biomarker such as a cell surface marker (e.g., CD34, CD117, and/or CD184) can be used to determine appropriate numbers of cells for stem cell therapy.

In aspect of the disclosure provides for cryopreservation and thawing of ATZ cells after harvesting ATZ crypts, single cells and/or after organoid culture by resuspending centrifuged cells in serum-free, DMSO-containing freezing medium and gradually decreasing the temperature to −80° C. or lower. To re-establish organoid cultures, cryopreserved ATZ organoids or single cells are quickly thawed in a 37° C. water bath, resuspended in pre-warmed tissue culture medium, and centrifuged and resuspended in fresh medium to remove freezing medium. In some embodiments, the composition further comprises cryopreservation media. In some embodiments, the ATZ or eATZ composition is cryopreserved.

### Methods of Treatment

In another aspect, the invention provides methods of treating a fistula in a subject in need thereof, the method comprising administering into the fistula an effective amount of the pharmaceutical composition of the ATZ cells described herein, or the dosage form, thereby treating the fistula. By way of example, an appropriate number of validated cells can be combined with a scaffold such as a scaffold paste for injection into a fistula. An exemplary scaffold paste is a collagen scaffold paste (Permacol™ Paste, Medtronic PLC, Minneapolis, MN). The effectiveness of this treatment is demonstrated in the following examples.

In some embodiments, the ATZ crypts are cultured ex vivo to produce expanded ATZ cells for use in a method of treating fistulas. In some embodiments, the eATZ cells can be cryopreserved after expansion and then thawed prior to treatment.

In some embodiments, the treatment causes the fistula to heal or close, for example, with new fibrotic tissue. In some embodiments, the fistula is a perianal fistula. In some embodiments, the fistula is a refractory fistula. The fistula can be associated with Crohn's disease or in an individual with a fistula of unknown aetiology.

In some embodiments, the pharmaceutically acceptable carrier comprises a biocompatible scaffold, for example, a scaffold described hereinabove.

### Pharmaceutical Compositions

In another aspect, provided herein are methods of preparing a therapeutic or pharmaceutical composition (e.g., a composition as disclosed herein). The method comprises (a) harvesting anorectal transition tissue from a patient or donor; (b) enzymatically digesting the transition tissue with an enzyme to prepare a cell suspension; (c) optionally combining at least a portion of the cell suspension with a cryopreservation media and cryopreserving the cell suspension; and (d) combining the cell suspension of step (b) or optional step (c) with a pharmaceutically acceptable carrier.

In one embodiment, the disclosure provides a pharmaceutical composition comprising adult allogeneic or autologous anorectal transition zone cells, or “ATZ cells”, in a pharmaceutically-acceptable carrier. The ATZ cells can be prepared by a method that comprises: (a) collecting anorectal transition tissue from an adult human or porcine subject; (b) preparing a cell suspension in vitro by enzymatic digestion of anorectal transition tissue; (c) sedimenting/pelleting and then re-suspending the cells in freezing medium and cryopreserving ATZ cells in, for example, liquid nitrogen. Prior to use, the cryopreserved ATZ cells are combined with a pharmaceutically-acceptable carrier, and the cell/carrier preparation is then injected into the fistula. The carrier can be used to fill the internal shape of the fistula, enabling tissue remodeling and more effective closure of the tract.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The following examples are merely illustrative and are not intended to limit the scope or content of the disclosure in any way.

### Example 1. Isolation of Intestinal Crypts from Anorectal Tissue

This example describes protocols for isolating intestinal crypts from anorectal tissue from a pig and a human subject with Crohn's disease.

### I. Pig

An example of healthy adult white Landrace porcine tissue from the colon to anus (FIG. 1(a)) was collected in AIMV V medium (Thermo Fisher Scientific) containing antibiotics/antimycotics within 2 hours of termination and processed directly, or in some instances after overnight storage in at 40 C. The tissue was longitudinally opened and cleaned.

The dentate line was identified between the rectal mucosa and anal skin (FIG. 1(b)). The width of the tissue was 8-10 cm with a height of 1-2 cm and was pale in colouration. The epithelial layer of dentate tissue (anorectal transition zone) was excised from rectal mucosa and anal skin (FIG. 1(c)). Enzymatic treatment of minced anorectal transition zone tissue released intestinal crypts (FIG. 1(d-g)) and structures consistent with submucosal glands (FIG. 1(h-i)).

### II. Crohn's Disease Patient

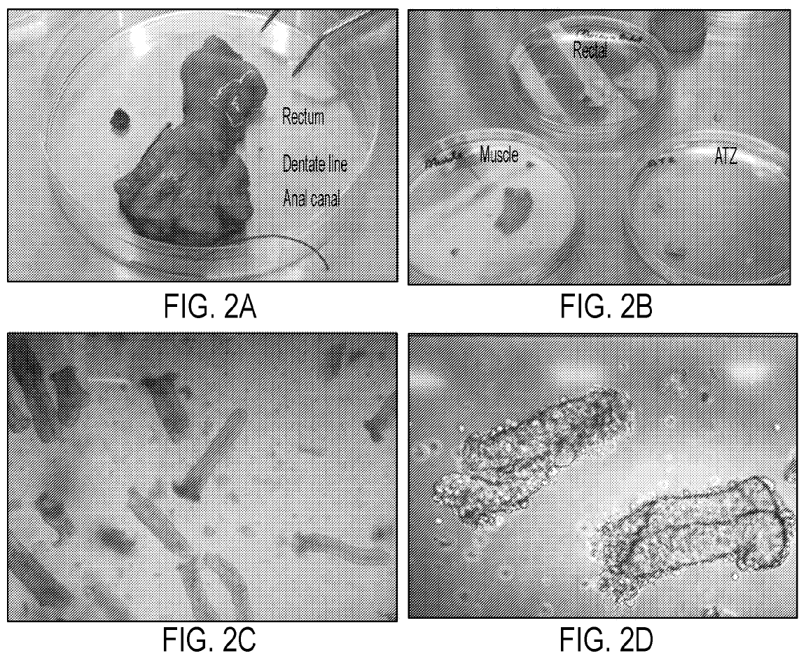

From a proctectomy tissue (FIG. 2(a)), the anorectal transition zone was excised (FIG. 2(b)), minced and enzymatically treated to release crypts (FIG. 2(c-d)).

### Example 2. Crypt Anorectal Transition Zone Organoid Development

Crypt anorectal transition zone organoid development from the pig and human tissue of Example 1 was developed.

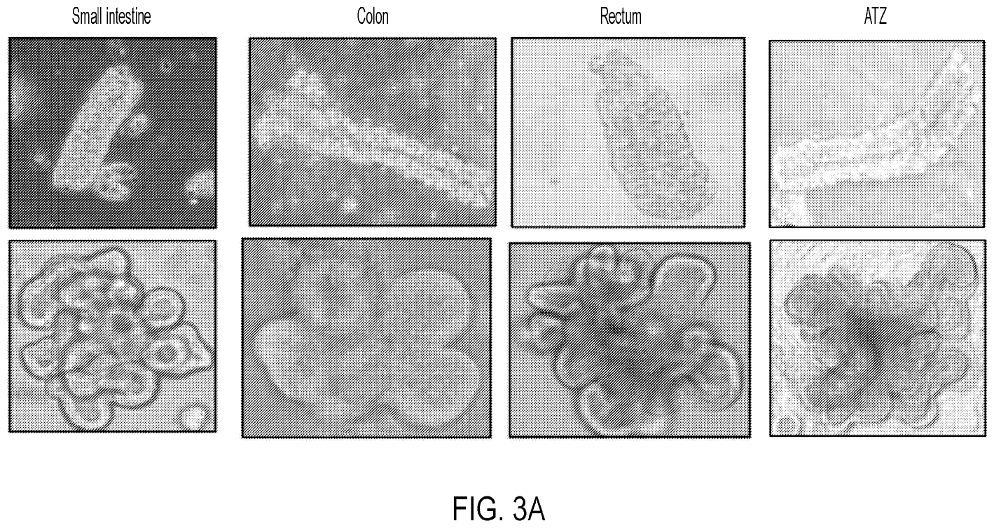

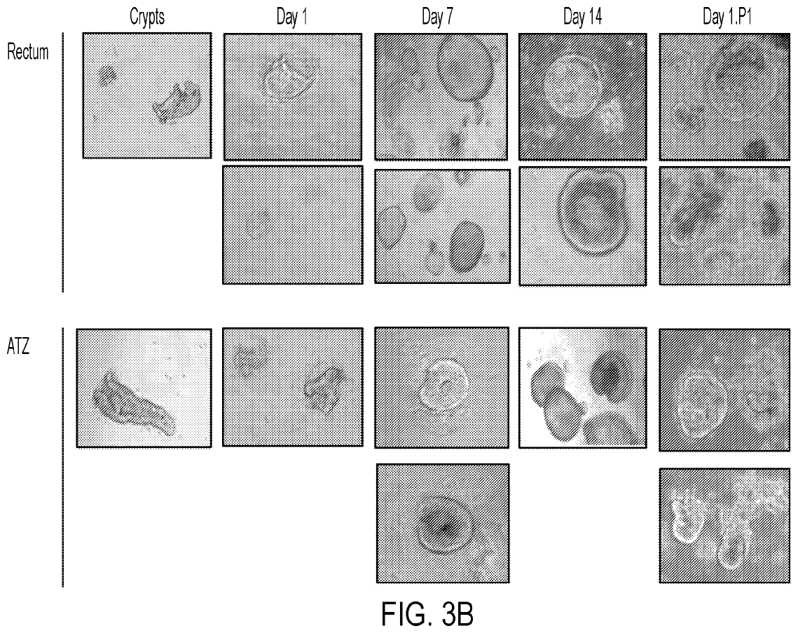

Intestinal crypts prepared in EXAMPLE 1 were embedded in Matrigel® Matrix (Corning, New York) with human organoid growth medium (OGM, IntestiCult™, STEMCELL Technologies, Inc. Vancouver, CA) and developed into fully branched organoids within 1-2 weeks. The morphology of porcine crypt organoids derived from anorectal transition zone were indistinguishable from organoids derived from porcine intestine, colon, and rectum tissue (FIG. 3(a)). Similarly, organoids derived from anorectal transition zone and rectum were indistinguishable in a Crohn's disease patient (FIG. 3(b)).

### Example 3. Increased Progenitor Capacity of Single Cell Preparations of Porcine ATZ Crypts Compared to Rectal Crypts

This example demonstrates that single cell preparations of porcine ATZ crypts compared to rectal crypts have increased progenitor capacity than rectal crypts.

The plating efficiencies (organoids formed per cells plated) of single cells preparations of porcine ATZ and rectal crypts were determined for an input of 500, 1,000 and 2,000 viable crypt cells per well in a 24 well flat well suspension culture plates (Sarstedt) and monitored weekly for 4 weeks. Organoid growth medium (IntestiCult™) was refreshed once at the end of week 2 without a change in Matrigel® Matrix.

Few organoids formed from 500 input cells in either group.

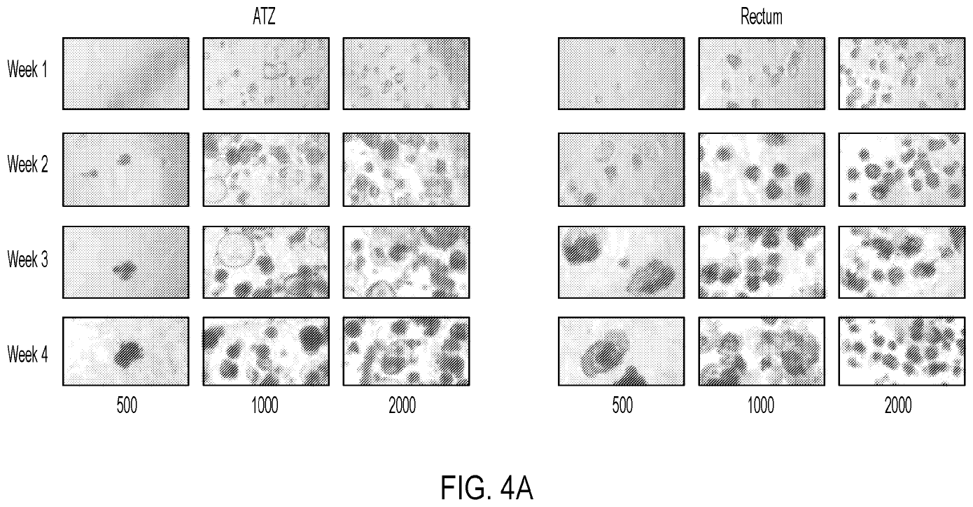

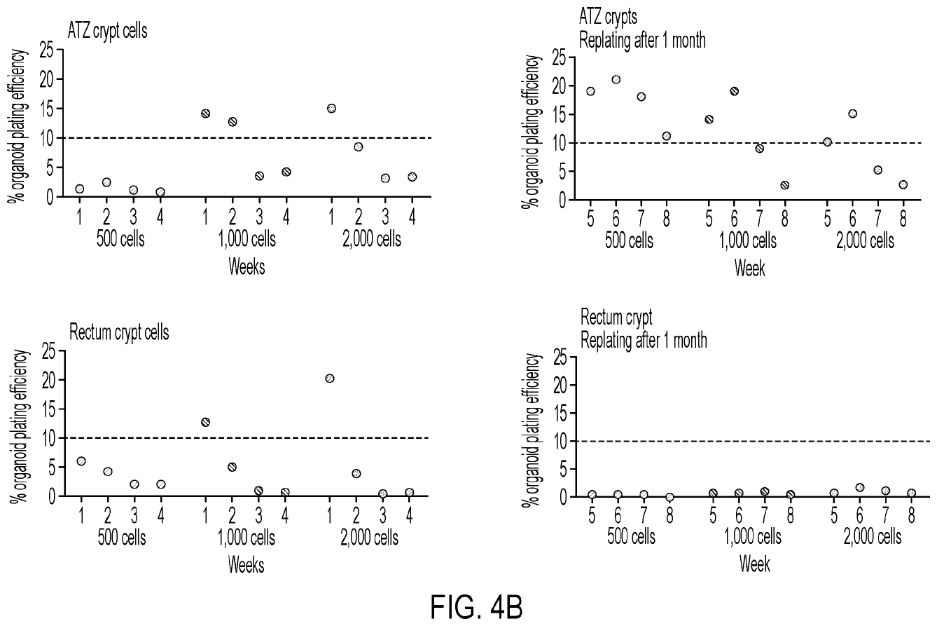

At week 1, the appearance (FIG. 4(a)) and frequency (FIG. 4(b)) of organoids forming was similar for ATZ and rectal organoids for cultures initiated with 1,000 or 2,000 cells. At week 2, rectal organoid cultures contained more fully differentiated organoids (dark clusters of cells), whereas ATZ organoid cultures contained more undifferentiated viable organoids (cystic structures) (FIG. 4(a)).

Few viable organoids were detected at weeks 3 and 4 in cultures initiated with 1,000 and 2,000 cells: 3-4% for ATZ and 0-1% for rectum (FIG. 4(b)).

To determine the progenitor capacity after 4 weeks, cultures were harvested and reinitiated with 500, 1,000, or 2,000 viable cells. OGM was refreshed at week 6 without a change in Matrigel® Matrix and organoid plating efficiencies were assessed at weeks 5, 6, and 7 (FIG. 4(b)). The highest plating efficiency was observed with 500 replated ATZ cells over weeks 5-7 (18%-21%). Plating efficiencies for 1,000 and 2,000 replated ATZ cells peaked at week 6 (19% and 15%, respectively) and decreased at week 7 (9% and 5%, respectively). Plating efficiencies for all replated rectal cells was <2% over weeks 5-7.

Taken together, these studies demonstrate an increased progenitor capacity in porcine ATZ crypt cells compared to rectum crypt cells, which is consistent with the existence of a long-term repopulating ATZ cell population.

### Example 4. Protein Expression of Stem Cell Markers of Embryonic Developmental Lineages on Freshly Isolated Crypts and Crypt Organoids

Protein expression of stem cell markers of embryonic developmental lineages on freshly isolated crypts and crypt organoids from pigs and a Crohn's disease patient for the purpose of describing and illustrating certain examples and embodiments of the present disclosure.

### Freshly Isolated Porcine Crypts

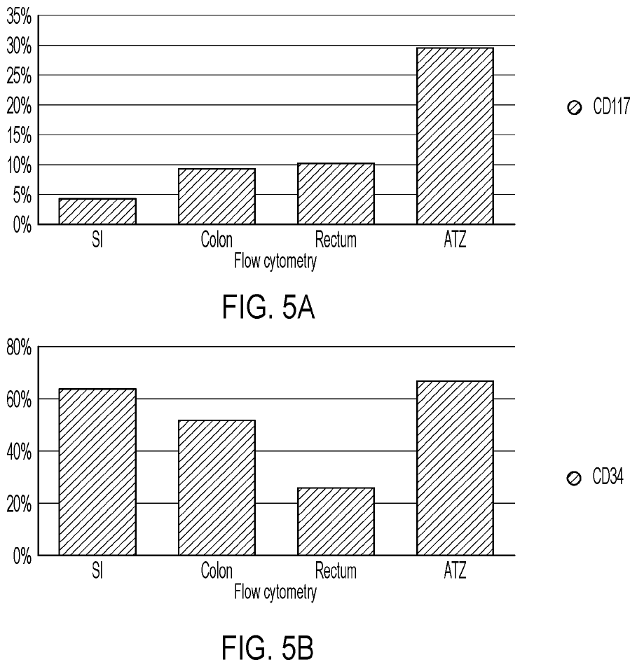

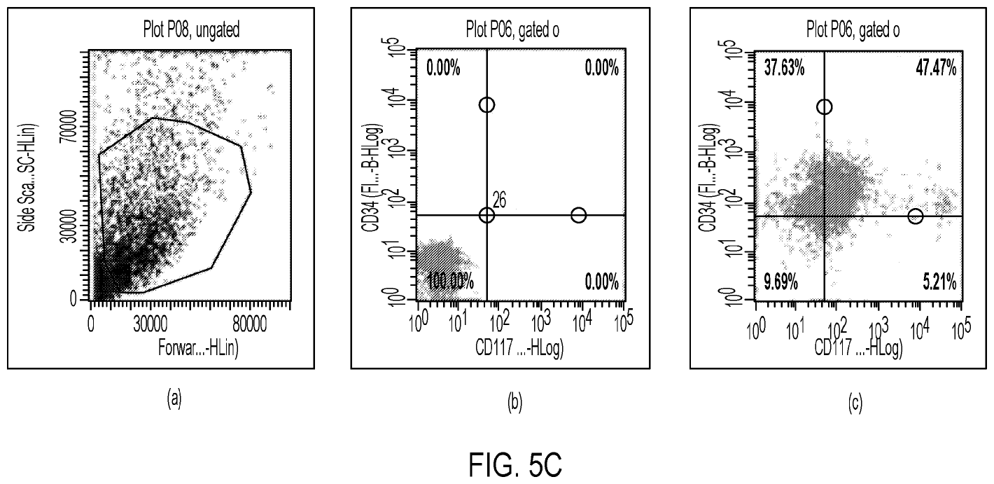

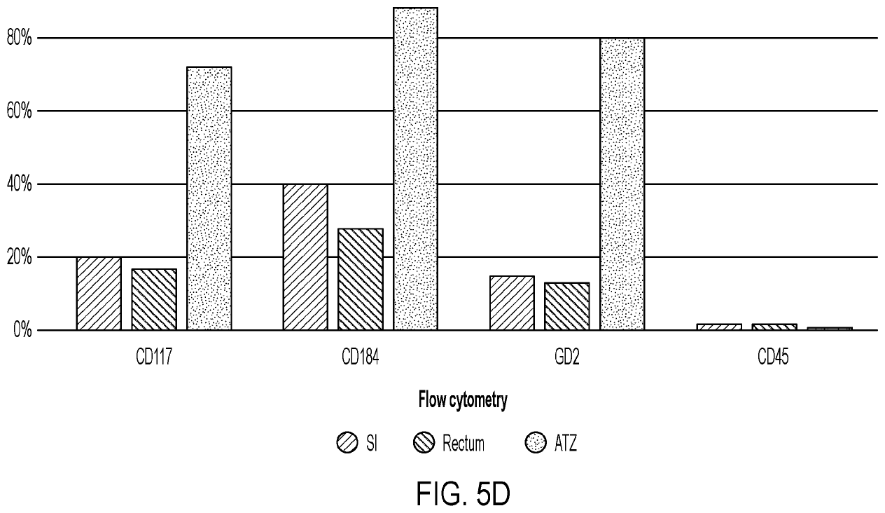

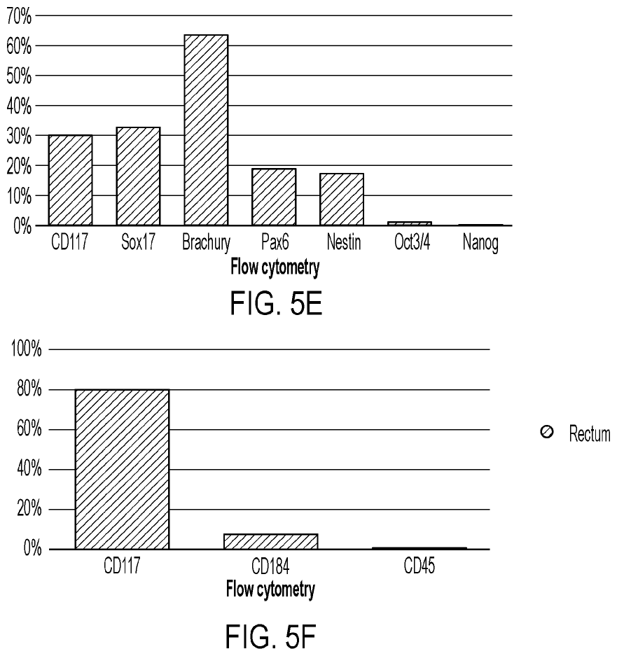

Cell surface stem cell markers were examined by flow cytometry on single cell preparations from freshly isolated crypts derived from the small intestine, colon, rectum, and anorectal tissue. Expression of the stem cell and progenitor cell growth factor receptor, KIT (CD117), was expressed at 3-5-fold higher levels in ATZ crypt cells compared to small intestine, colon, and rectum crypt cells (FIG. 5(a)). Crypt cells from these tissues all expressed the stem cell and progenitor cell marker CD34 to varying extent, with ATZ crypt cells expressing the highest levels (FIG. 5(b)).

In a double labelling experiment, 85% of ATZ cells expressed CD34 and 53% of cells expressed CD117; 47% of cells co-expressed CD34 and CD117; of the CD117 expressing cells, 88% co-expressed CD34 (FIG. 5(c))

### Porcine Crypt Organoids

Cell surface stem cell markers were examined by flow cytometry on single cell preparations of fully differentiated porcine organoids derived from the small intestine (SI), rectum, and anorectal tissue after 10 days of culture. Expression of KIT (CD117), CXCR4 (CD184), and GD2 were increased by more than 3-fold in ATZ organoids compared to SI and rectal organoids. The hematopoietic marker, CD45, was undetected in all crypt organoids (FIG. 5(d)).

Since KIT and CXCR4 are markers of definitive endoderm and GD2 is a marker of primitive mesoderm, further investigation of the developmental lineage origins of freshly isolated ATZ crypt cells was undertaken. Intracellular protein markers confirmed the expression of transcriptional factors associated with the endoderm (SOX17), mesoderm (BRACHURY), and ectoderm (PAX6 and NESTIN) lineages. However, expression of transcriptional factors associated with pluripotency (OCT4 and NANOG) were not detected by flow cytometry (FIG. 5(e)).

Taken, together these results indicate porcine ATZ crypt cell populations express stem cell and progenitor protein markers for all 3 developmental lineages, and that markers for endoderm and mesoderm were maintained in ATZ crypt organoids.

### Crohn's Patient Rectal Crypt Organoids

Cell surface expression of CD117 was detected at 80% in a single cell preparation of rectal crypt organoids of a Crohn's patient (FIG. 5(f)).

### Example 5. mRNA Expression Profiling of Porcine ATZ Crypt Cells

This example shows mRNA expression profiling of porcine ATZ crypt cells.

### Fresh Porcine ATZ Crypts

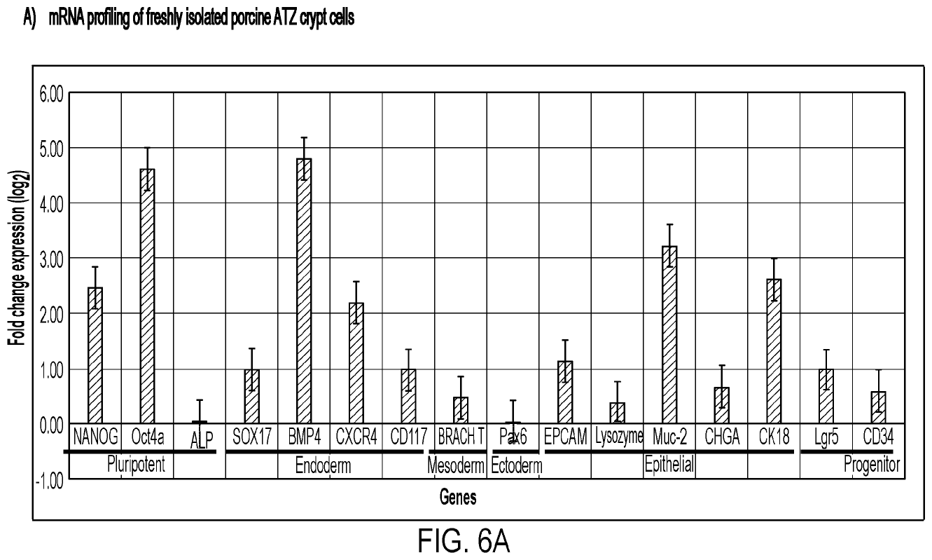

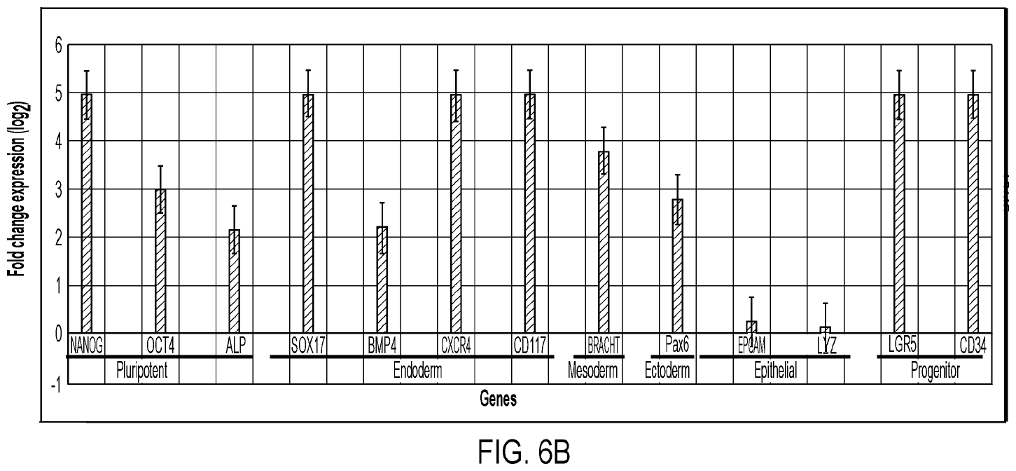

Transcriptional profiling of fresh porcine ATZ crypt cells confirmed basal expression of markers for mature epithelial cells (EPCAM, LYZ, MUC2, CHGA, CK18); stem cells and progenitor cells (CD34, LGR5); and developmental stem cells of the endoderm, mesoderm (BRACHURY), and ectoderm (PAX6) lineages; and pluripotent stem cells (NANOG, OCT4A) (FIG. 6(a)). Although mRNA expression of alkaline phosphatase (ALP) was not detected, the enzyme was detected by immunocytochemistry in Example 9.

### Porcine ATZ Crypt Organoids.

Transcriptional profiling of ATZ crypt cells cultured in organoid growth medium (IntestiCult™) retained mRNA expression for markers of stem cells of all three developmental lineages. (FIG. 6(b)). Lower levels of mRNA expression for markers of mature epithelial cells were detected when compared to fresh ATZ cells. mRNA expression levels were normalised to glyceraldehyde 3-phosphate dehydrogenase (GAPDH).

Taken together, transcriptional profiling of fresh ATZ crypts and organoids confirmed the presence of markers for stem cells of all three developmental lineages.

### Example 6. Culture Medium for Maintaining and Expanding Multipotent Porcine Anorectal Transition Stem Cells

This example demonstrates in vitro culture conditions for maintaining and expanding multipotential ATZ cells.

Freshly isolated porcine ATZ crypt single cells were cultured for 14 days in either (1) a feeder-free medium that maintains human embryonic stem cells (ESCs) and induced pluripotent stem cells (iPSCs) in an undifferentiated state (mTeSR™, STEMCELL Technologies, Inc. Vancouver, CA)); or in (2) standard crypt organoid growth medium (IntestiCult™).

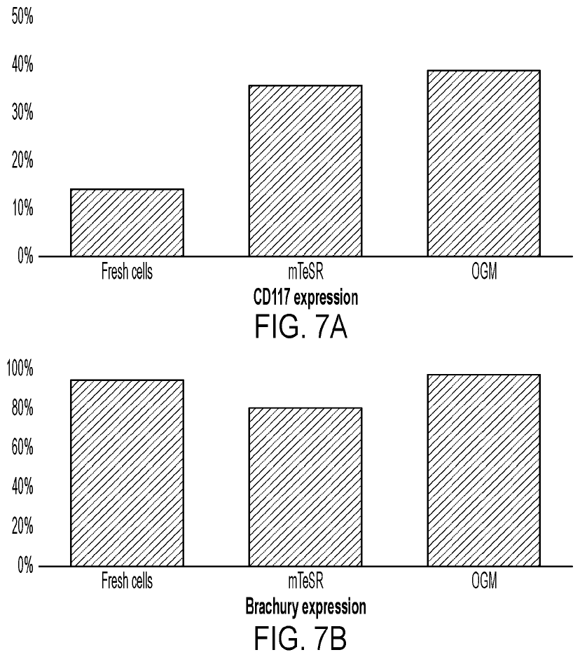

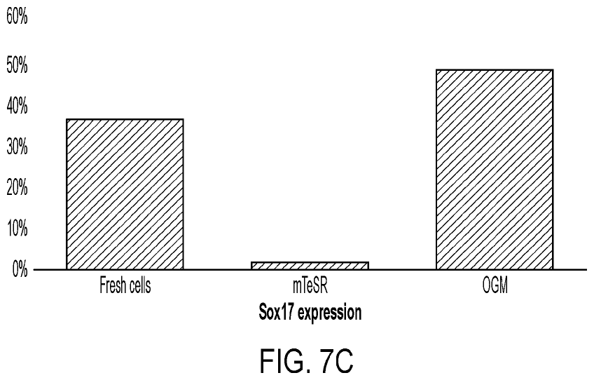

The frequency of multipotential stem cell markers described in Example 4 were assessed by flow cytometry before culture (day 0) and after 14 days in culture. The stem cell and progenitor marker CD117 was expressed at 14% in ATZ crypt cells before culture (day 0) and increased to 35% in mTeSR and 28% OGM at day 14 (FIG. 7(a)). The intracellular marker for mesoderm, BRACHYURY, was expressed at 97% ATZ crypt cells before culture (day 0) and the expression levels were maintained at 99% in mTeSR and 81% in OGM at day 14 (FIG. 7(b)). The intracellular marker for endoderm, SOX17, was expressed in 34% of ATZ crypt cells before culture (day 0) and <1% expression in mTeSR medium and 49% in OGM at day 14 (FIG. 7(c)).

These results indicate that OGM may be better for maintaining ATZ crypt cells expressing markers of both endoderm and mesoderm developmental lineages.

### Example 7. In Vitro Differentiation of Porcine Anorectal Transition Cell-Derived Crypts into all Three Developmental Lineages

This example demonstrates that porcine anorectal transition cell-derived crypts differentiate in vitro into all three developmental lineages (endoderm, mesoderm, and ectoderm).

Expression of protein and mRNA markers in all three developmental lineages of fresh porcine ATZ crypt stem cells in Example 4 and Example 5, respectively, led to studies to determine if freshly isolated ATZ crypt cells had the capacity to generate mature cells of the endoderm lineage (gut mucosa), mesoderm (blood vessels), and ectoderm (keratinocytes).

### Example 7.1. Endoderm Differentiation Potential of Fresh Porcine ATZ Crypts

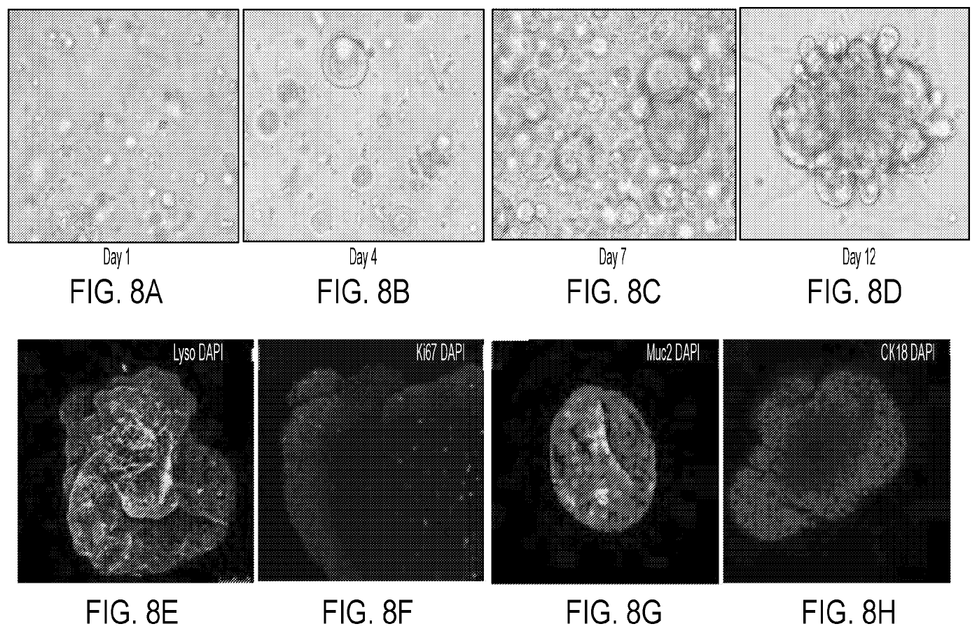

Protein expression levels of mature endoderm cell markers of gut mucosa were assessed after two weeks of culturing single cell preparations of fresh ATZ crypts embedded in Matrigel® Matrix with OGM to generate organoids. Immunostaining of intact organoids was visualised for expression of LYSOZYME, proliferating cells (KI67), secretory cells (MUC2), and cytokeratin 18 (CK18) which is expressed in the single layer of gut epithelium (FIG. 8(a-h)).

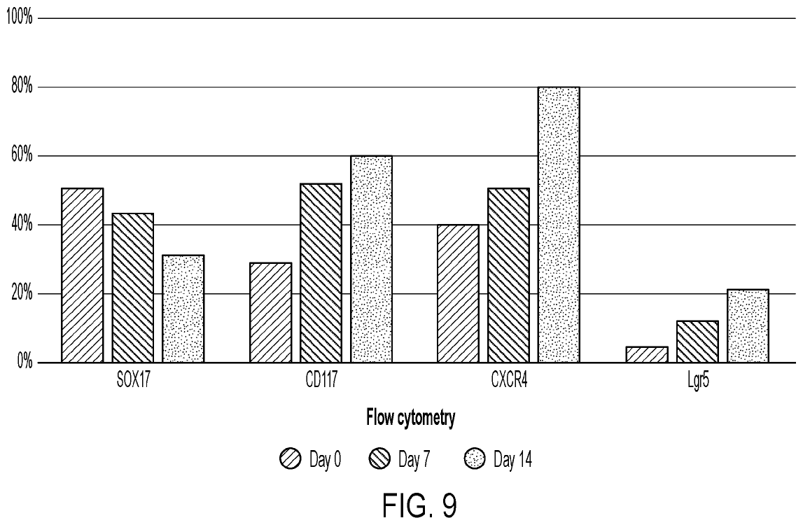

Flow cytometric analysis of fresh ATZ crypt cells showed that SOX17 decreased from 52% at day 0 to 34% at day 14; and that KIT (CD117) and CXCR4 (CD184) increased by twofold during culture (FIG. 9). The intestinal stem cell marker, LGR5, was expressed at 3% on day 0 and increased to 22% at day 14.

Taken together these results support that fresh ATZ crypts have the capacity to generate mature cell types derived from the endodermal lineage.

### Example 7.2. Mesoderm Differentiation Potential of Porcine ATZ Crypts

Protein expression levels of mature mesoderm cell markers were assessed after two weeks of culturing single cell preparations of fresh ATZ crypts in MethoCult™ to test their capacity to generate progeny in the haematopoietic lineage. No haematopoietic colonies formed in the non-adherent, methylcellulose layer.

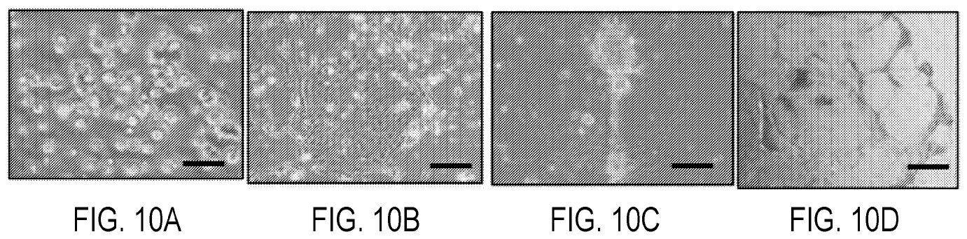

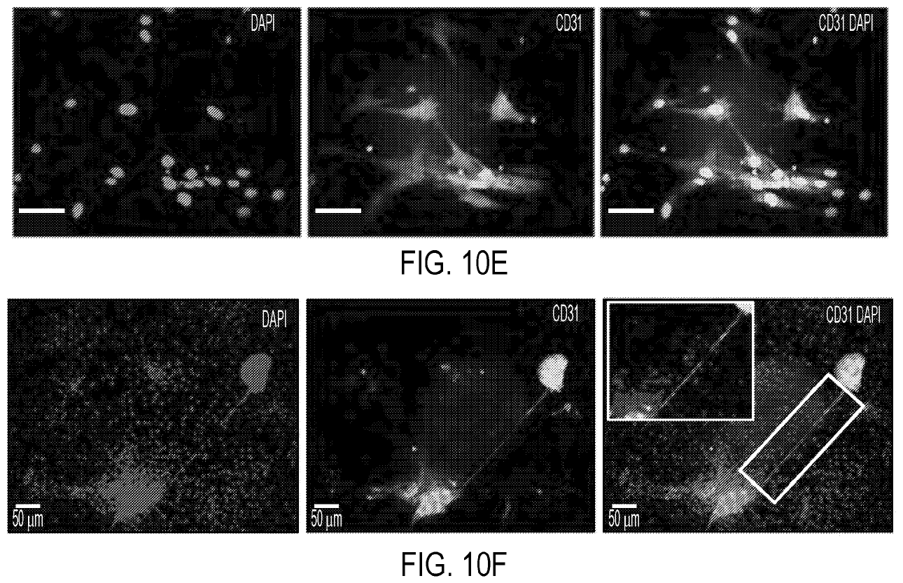

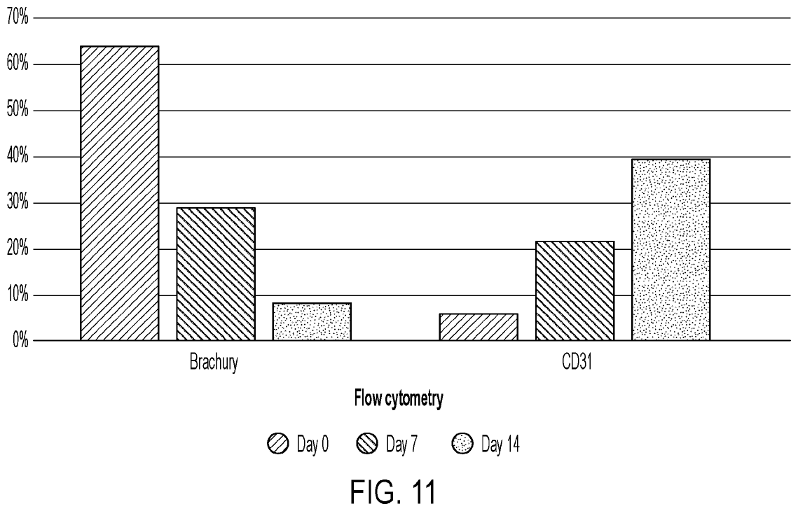

However, single ATZ crypt-derived cells (FIG. 10(a)) formed clusters in the adherent layer at day 3 (FIG. 10(b)) and initiated and formed a network of blood vessel-like tubular structures at day 10 (FIG. 10(c-d)). Immunostaining with the endothelial intracellular marker, CD31 (PECAM), was detected at the initiation and formation of a network of blood vessel-like tubular structures (FIG. 10(e-f)). Flow cytometric analysis of fresh ATZ cells showed that BRACHURY expression decreased from 67% at day 0 to 23% at day 7 and decreasing further to 7% on day 14; CD31 expression increased from 5% at day 0 to 19% and further at day 7 to 37% at day 14 (FIG. 11).

Taken together these results support that fresh ATZ crypts have the capacity to generate mature cell types derived from the mesoderm lineage.

### Example 7.3. Ectoderm Differentiation Potential of Porcine ATZ Crypts

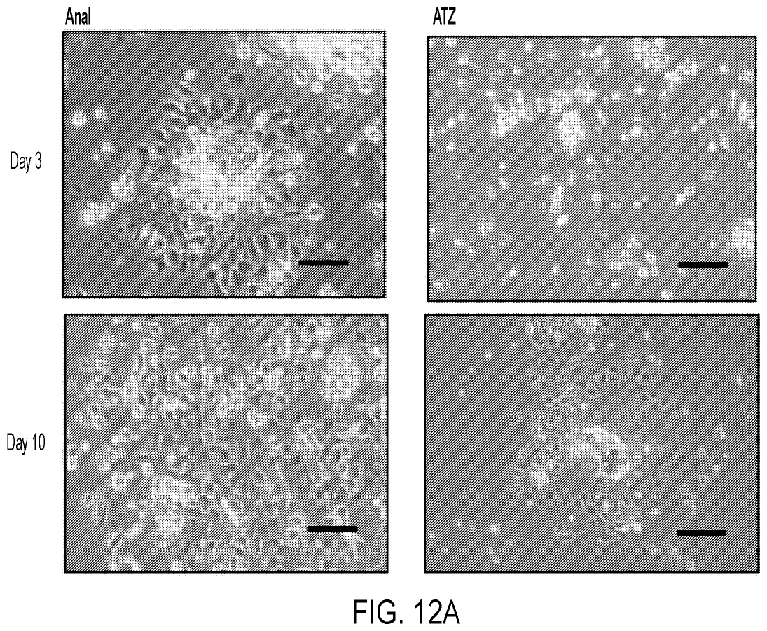

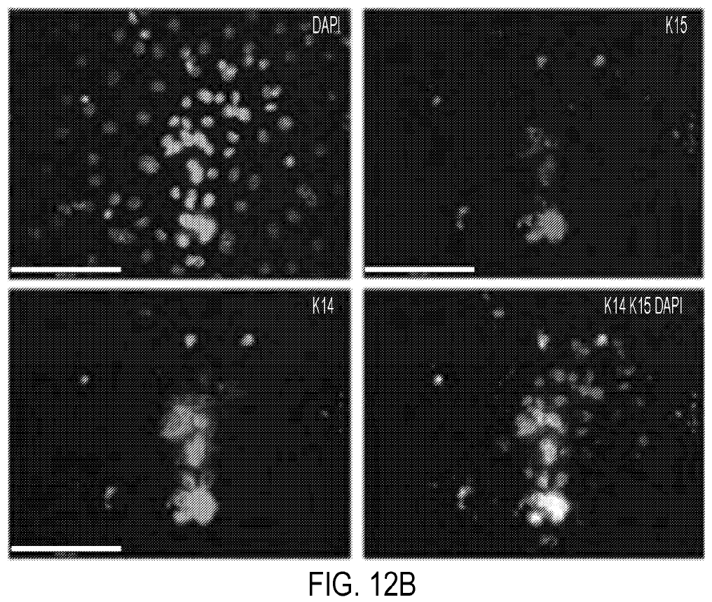

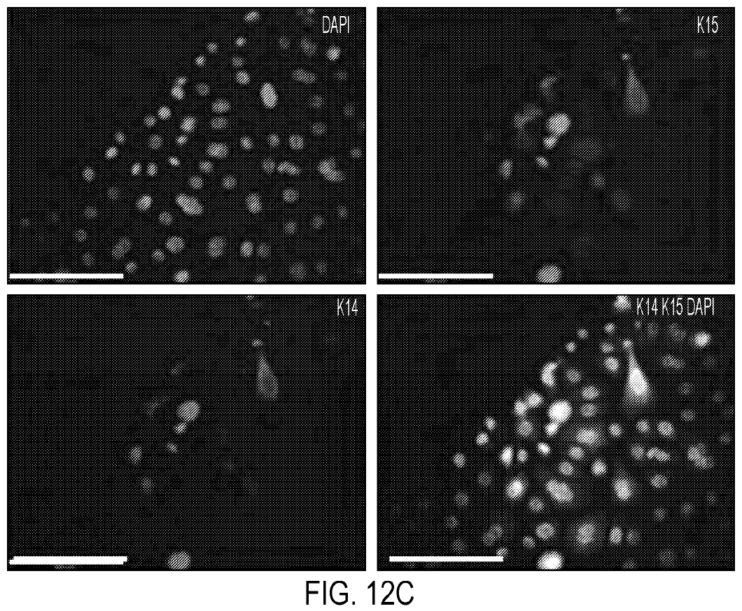

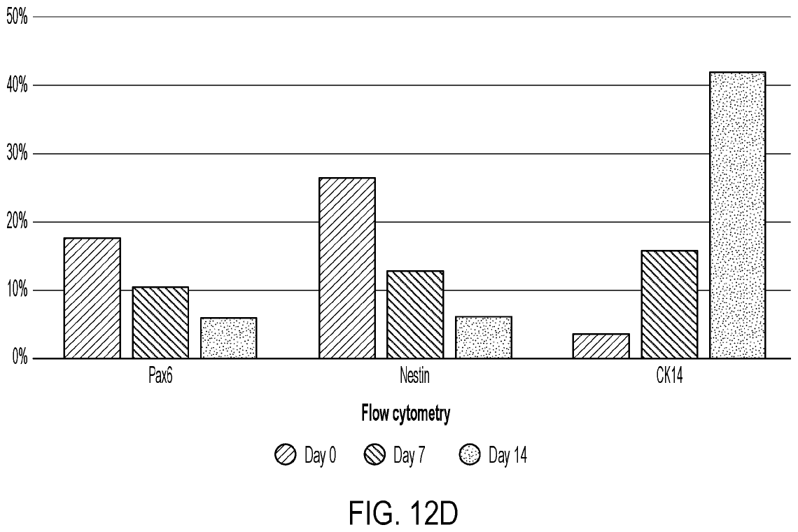

Protein expression levels of mature ectoderm cell markers were assessed after two weeks culturing single cell preparations of fresh ATZ crypts and anal skin in keratinocyte differentiation medium (KSFM, Sigma-Aldrich, St. Louis, MO). Cell clusters developed at day 3 and a cobblestone-like adherent layer with keratinocyte morphology formed in anal skin and ATZ cell cultures by day 10 (FIG. 12(a)). Co-expression of the keratinocyte markers, CK14 and CK15, were detected by immunostaining of anal skin adherent cultured cells (FIG. 12(b)) and ATZ adherent cultured cells (FIG. 12(c)).

Flow cytometric analysis of ATZ adherent culture cells showed that the ectodermal lineage marker, Pax 6, expression decreased in culture from 18% at day 0 to 6% at day 7 and 5% at day 14 (FIG. 12(d)). CK14 expression on ATZ adherent culture cells increased over time in culture from 3% on day 0 to 18% on day 7 and 42% on day 14 (FIG. 12(d)).

Taken together these results support that fresh ATZ crypts have the capacity to generate mature cell types derived from the ectoderm lineage.

### Example 8. Absence of Multipotential Stem Cells of all Three Developmental Lineages in Porcine Small Intestine Crypts

This Example demonstrates that porcine small intestine (SI) crypts have the potential to generate mature cells of the endodermal, mesodermal, and ectodermal lineages in vitro using the same methods as described in Example 7 for porcine ATZ crypts.

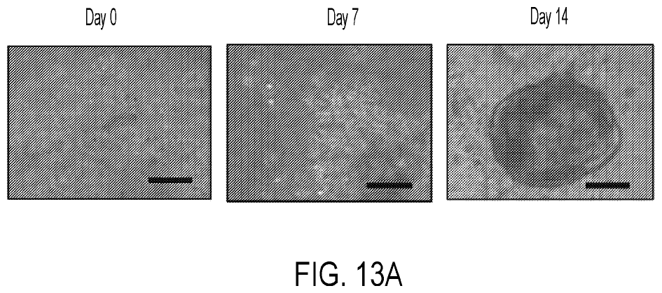

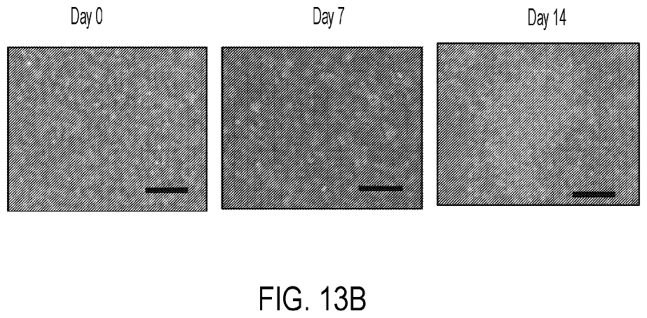

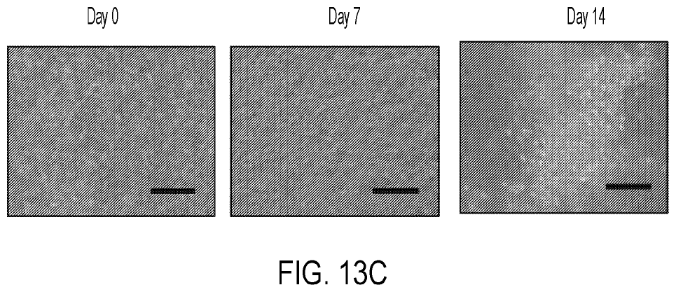

SI single crypt cells were cultured for two weeks in organoid growth medium (IntestiCult™) promoting differentiation to endoderm, mesoderm (MethoCult™) and ectoderm (KSFM, Sigma-Aldrich, St. Louis, MO). SI crypt cells generated organoids OGM, as expected (FIG. 13(a). However, no growth was detected for SI crypt cells at days 7 or 14 when cultured in MethoCult (FIG. 13(b)) or KFSM (FIG. 13(c)).

Taken together, these results are consistent with the absence of multipotential stem cells in porcine SI crypts capable of generating cell types of all three developmental lineages, using the assays demonstrated for ATZ crypt cells.

### Example 9. In Vitro Embryoid Body Assay to Assess the Pluripotency of Porcine ATZ Crypt Cells

This Example demonstrates that single cell preparations of fresh ATZ crypts cultured in feeder-free mTeSR™ medium can promote embryoid body formation.

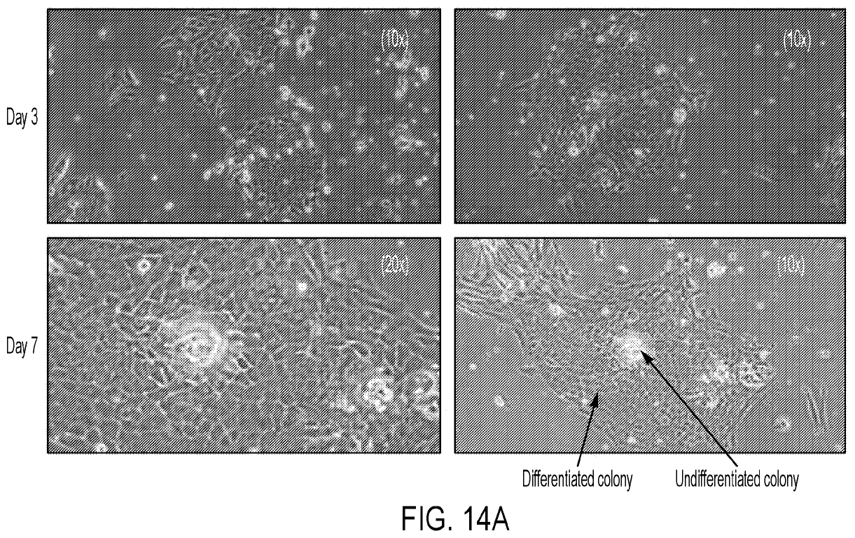

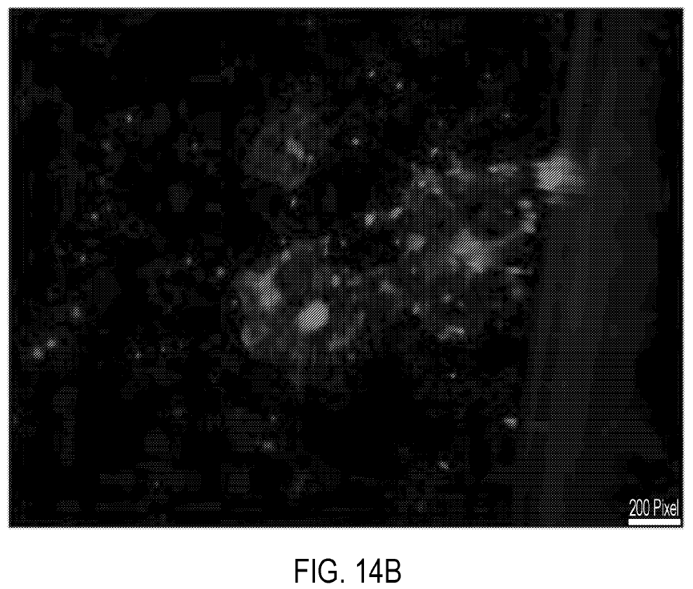

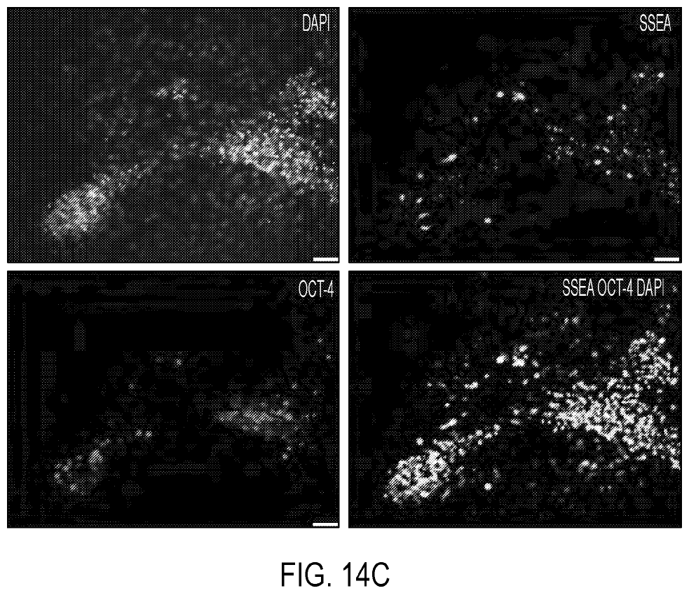

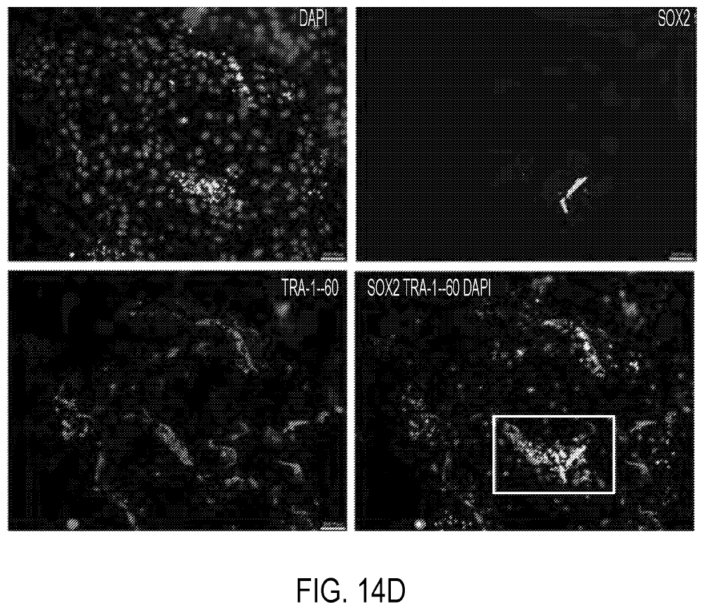

Fresh ATZ crypt single cells plated at a high density generated a cobblestone-like adherent layer by day 3; colonies of undifferentiated adherent cells developed by day 7; and differentiated adherent cells were generated and migrated away from the undifferentiated cell colonies (FIG. 14(a)).

Visualisation of alkaline phosphatase staining confirmed stem cell-like properties consistent with embryoid bodies (FIG. 14(b)). Furthermore, pluripotent stem cell markers, OCT4 and SSEA4 (FIG. 14(c)) and SOX2 and TRA-1-60 (FIG. 14(d)) were detected by immunocytochemistry at day 5.

Taken together, the results of the in vitro embryoid body assay are consistent with ATZ crypt cells exhibiting pluripotent stem cell-like properties.

### Example 10. Compatibility of Porcine ATZ Crypt Cell Growth with a Synthetic Scaffold

This example demonstrates the biocompatibility of porcine ATZ crypt cell growth in OGM with a synthetic scaffold.

Single cell preparations of fresh porcine ATZ crypt cells were cultured in OGM with 2%, 5%, or 10% synthetic non-functionalised scaffold (“PG” or PeptiGel® Alpha 2, Manchester BioGel, Chesire UK) and compared to standard Matrigel® Matrix cultures.

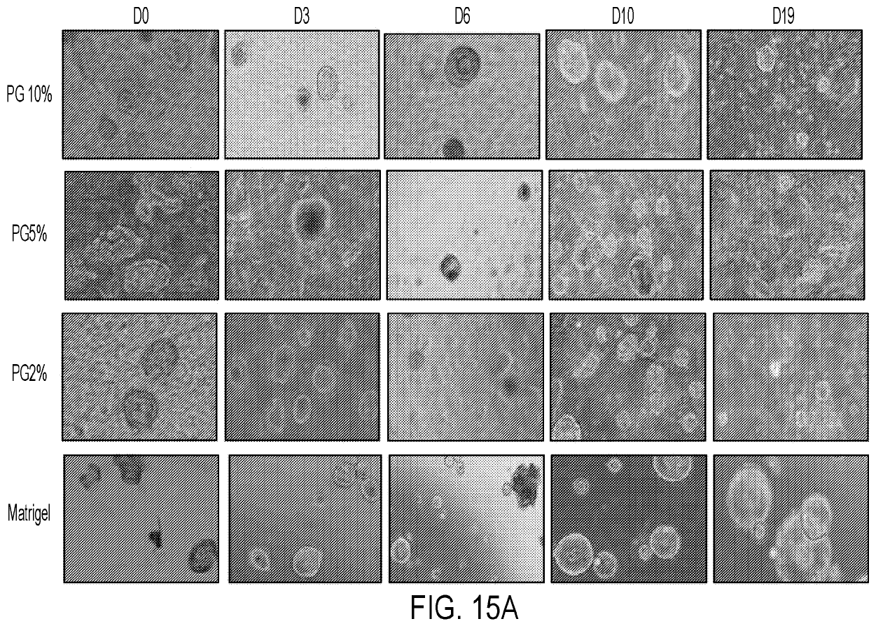

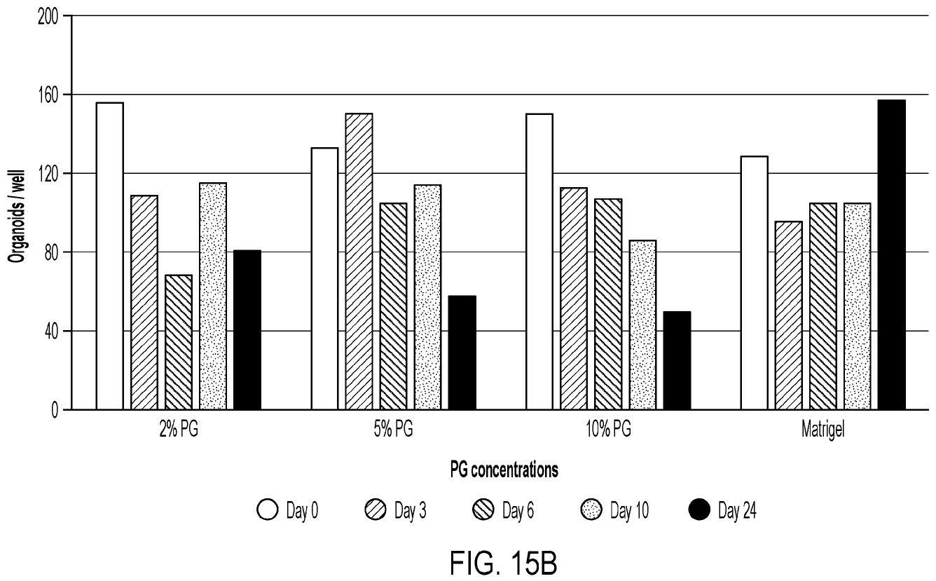

Similar appearing crypt organoids were generated in all concentrations of PGs and Matrigel® Matrix up to day 19 (FIG. 15(a)). However, the number of viable organoids in PGs decreased relative to OGM at day 24 (FIG. 15(b)), indicating that Matrigel® Matrix provided additional growth factors for stem cell and progenitor cell growth.

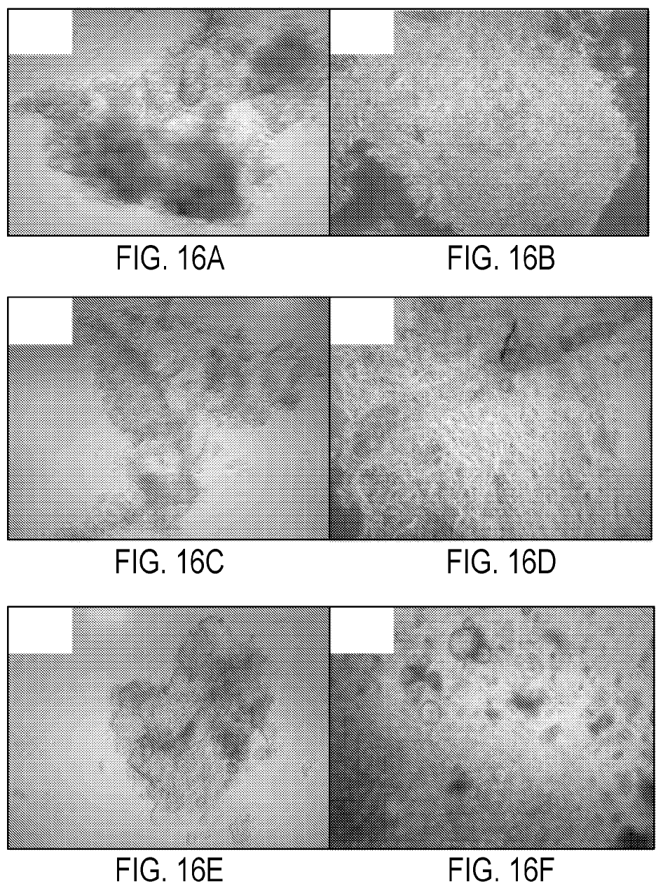

A range of functionalised Alpha 4 PGs were assessed for activity contributed by the binding domains: non-functionalised (FIG. 16(a)), fibronectin (RGD) (FIG. 16(b)), collagen (GFOGER) (FIG. 16(c)), and laminin (IKVAV, YIGSR) (FIG. 16(d-e)). An increased number of ATZ crypt organoids were associated with laminin functionalised PGs in liquid culture (FIG. 16(d-e)), similar to that of Matrigel® Matrix (FIG. 16(f)).

Based on these studies scaffold biocompatibility results with ATZ crypt cells. Equal volumes PGs functionalised with IKVAV and YIGSR were used as the pharmaceutical carrier for the in vivo studies described in Example 11.

### Example 11. Preclinical Porcine Fistula Model to Assess a Hydrogel Scaffold to Support Allogeneic Adult Porcine ATZ Cells to Heal Fistulas

This Example demonstrates that allogenic adult porcine ATZ cells in a hydrogel scaffold can be used to treat an anal fistula.

A validated preclinical porcine fistula model previously developed to assess surgical and sealant treatments of perianal fistulas) (Himpson et al. (2009) “An experimentally successful new sphincter-conserving treatment for anal fistula,” Dis. Colon Rectum, 52(4): 602-608) was used to test the safety and efficacy of treating fistulas with allogeneic adult porcine ATZ cells.

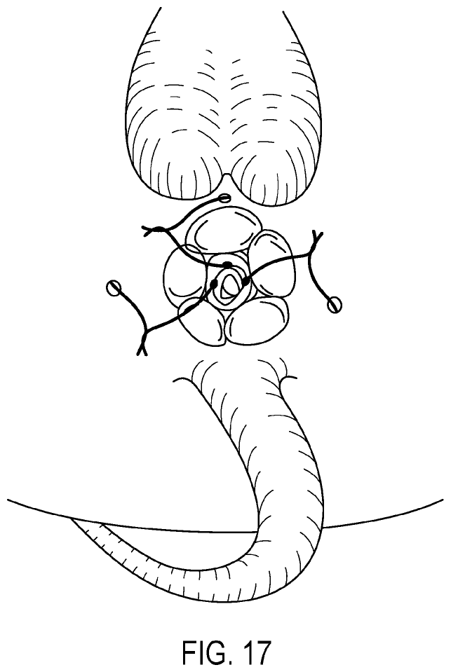

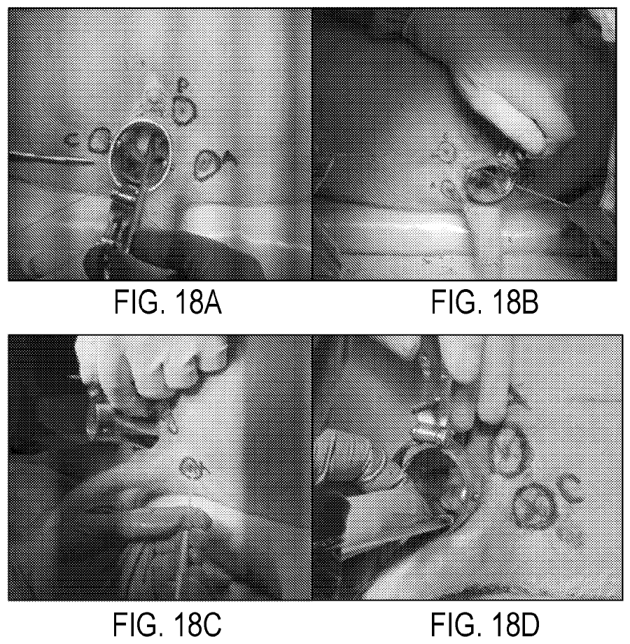

Three fistulas were mechanically created in each female white Landcross pig from the lumen of the anorectal region through muscle and fat to the outer skin (FIGS. 17 and 18). Threads or “setons” were inserted into the fistulas to promote chronic inflammation. After one month, the setons were removed and the fistula tracts were cleaned to remove granulation tissue before treatment.

Cell preparations of allogeneic porcine ATZ crypts from three pigs were harvested as described in Example 1 and cryopreserved in cryopreservation media (CryoStor®, STEMCELL Technologies, Inc., Vancouver, Canada) in advance of the study. The viability of cryopreserved ATZ cells was confirmed in advance of the study using the assay described in Example 10.

The treatment groups for each pig included: (1) No treatment control; (2) pharmaceutical carrier alone (PG-IKVAV and PG-YIGSR where “PG”=PeptiGel®) or “PG scaffold”; and (3) ATZ cells+ “PG scaffold” (TABLE 1). The study was terminated at day 90. Anorectal tissues were collected in 10% neutral buffer formalin (NBF). Paraffin-embedded tissues were sectioned for histology and stained with haematoxylin and eosin (H&E) and picrosirius red (PSR).

TABLE 1

| Subject | | Volume | |
|---|---|---|---|
| No. | Treatment | (mL) | # of Cells |
| 1 | No treatment control | None | None |
| | PG scaffold | 0.5 | None |
| | ATZ cells + PG scaffold | 0.8 | 10.8 million |
| 2 | No treatment control | None | None |
| | PG scaffold | 0.8 | None |
| | ATZ cells + PG scaffold | 0.8 | 30 million |
| 3 | No treatment control | None | None |
| | PG scaffold | 0.9 | None |
| | ATZ cells + PG scaffold | 0.8 | 75 million |

### Example 12. Histological Evidence that Allogeneic Adult Porcine ATZ Cells have Anti-Inflammatory Properties and Enable Tissue Remodeling for More Effective Closure of the Fistula Tract

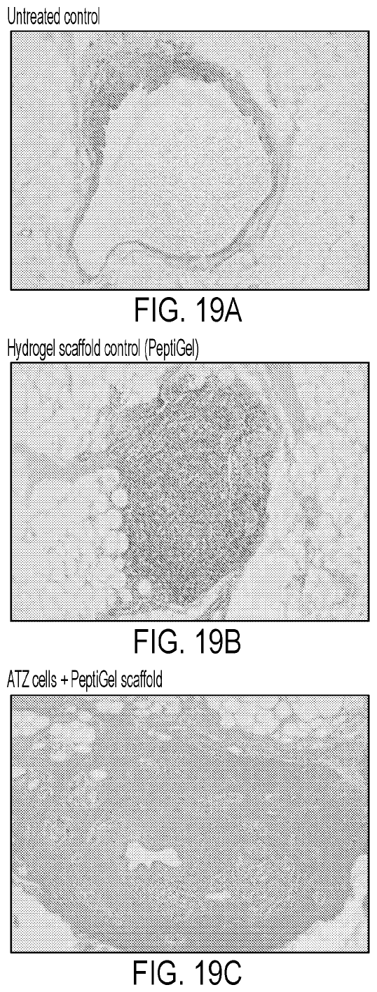

This Example demonstrates that, by histological analysis of tissues from porcine fistula tracts in Example 11, tissue remodeling leads to closure (healing) of the fistula tract.

Where treatment was withheld, the luminal surface of the fistula tract within fat tissue was lined with fibroblast-like cells, indicating that the luminal void would likely not close over time (FIG. 19(a)).

For treatment of fistula tract with the PG scaffold alone, the fistula tract within fat tissue was filled with inflammatory cells at day 90, indicating that the PG scaffold itself elicited a chronic inflammatory response that persisted throughout the treatment period with no evidence of tissue remodeling (FIG. 19(b)).

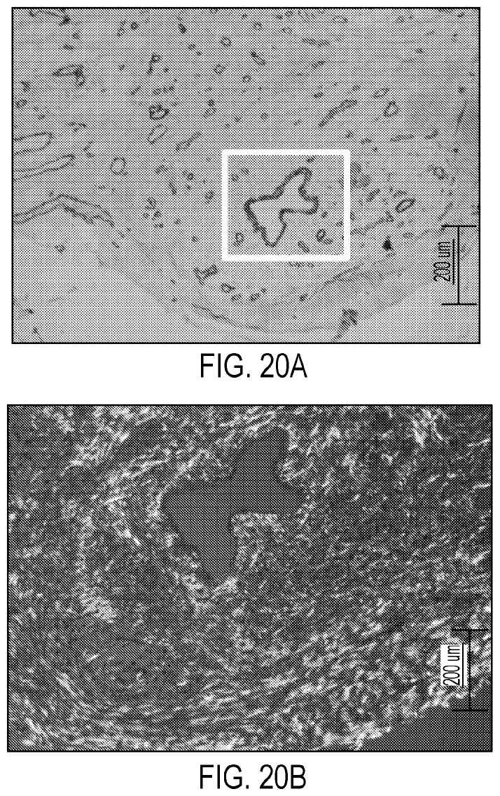

Treatment of the fistula tract with ATZ cells administrated with the PG scaffold effectively filled the tract within fat tissue with remodeled fibrotic tissue containing immature blood vessels, fibroblasts, smooth muscle cells and signs of immature adipose tissue (FIG. 19(c) and FIG. 20).

Taken together, this in vivo study provided histological evidence that adult allogeneic ATZ stem cells and progenitor cells (1) reduced PeptiGel® scaffold-mediated chronic inflammation, and (2) substantially reduced of the fistula tract opening with vascularized, remodeled tissue.

Porcine ATZ cells in a pharmaceutical carrier demonstrate anti-inflammatory properties in the porcine preclinical model similar to the anti-inflammatory properties of human adipose derived mesenchymal stem cells (AdMSCs, Alofisel™) used to treat Crohn's fistulas.

However, ATZ cells in a pharmaceutical carrier in this validated preclinical model closed the fistula tract at day 90, whereas Alofisel™ treatment of Crohn's patients only delays the time between fistula cleanings.

### Example 13. Preclinical Porcine Fistula Model to Assess a Collagen Scaffold to Support Gender Mismatched Allogeneic Adult Porcine ATZ Cells to Heal Fistulas

This Example demonstrates that allogeneic adult porcine ATZ cells in a collagen scaffold can be used to treat an anal fistula.

Cell preparations of allogeneic porcine ATZ crypts from one male pig were harvested as described in Example 1 and cryopreserved in cryopreservation media (CryoStor®, STEMCELL Technologies, Inc., Vancouver, Canada) in advance of the study. The viability of cryopreserved ATZ cells was confirmed in advance of the study using the assay described in Example 10.

Three fistulas were mechanically created in one white female Landcross pig from the lumen of the anorectal region through muscle and fat to the outer skin as in Example 11 (FIG. 17). Setons were inserted into the fistulas to promote chronic inflammation. After one month, the setons were removed and the fistula tracts were cleaned to remove granulation tissue before treatment.

The treatment groups for the pig included: (1) No treatment control; (2) pharmaceutical carrier alone (Permacol™); and (3) ATZ cells+Permacol (TABLE 2). The study was terminated at day 63. Paraffin-embedded tissues were sectioned for histology and stained with haematoxylin and eosin (H&E) and picrosirius red (PSR).

TABLE 2

| Subject | | Volume | |
|---|---|---|---|
| No. | Treatment | (mL) | # of Cells |
| 1 | No treatment control | None | None |
| | Permacol scaffold | 0.8 | None |
| | ATZ cells + Permacol | 0.8 | 8 million |
| | scaffold | | |

### Example 14. Histological Evidence that a Collagen Scaffold Supports Gender Mismatched Allogeneic Adult Porcine ATZ Cells Tissue Remodeling for More Effective Closure of the Fistula Tract

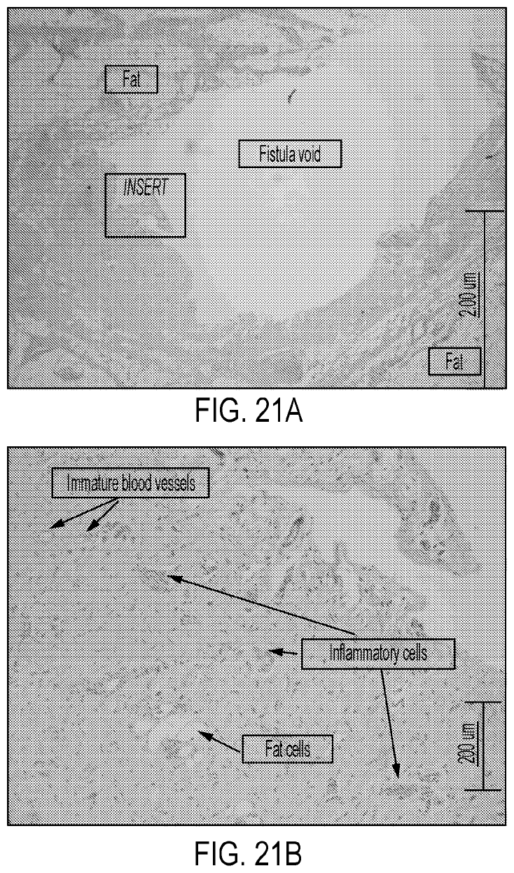

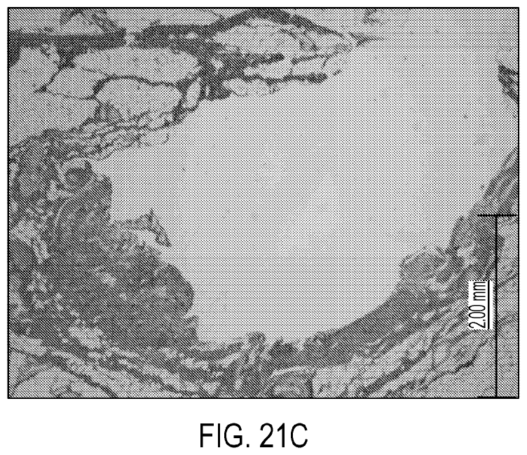

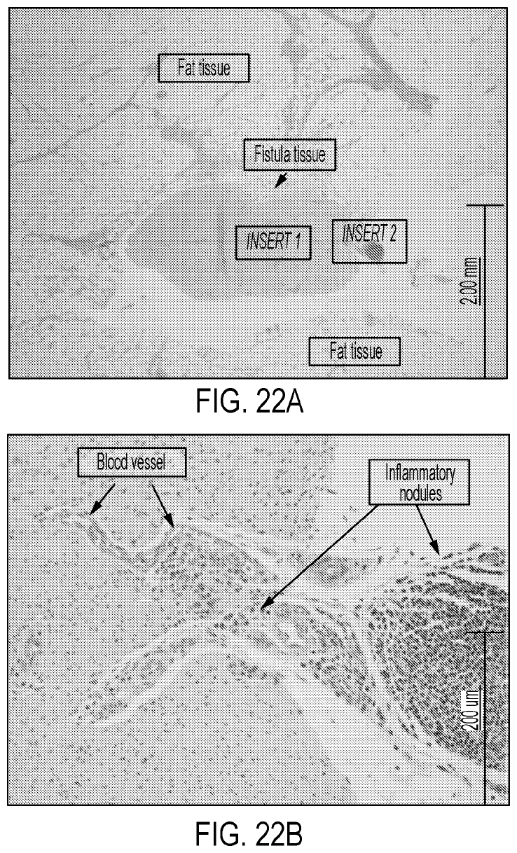

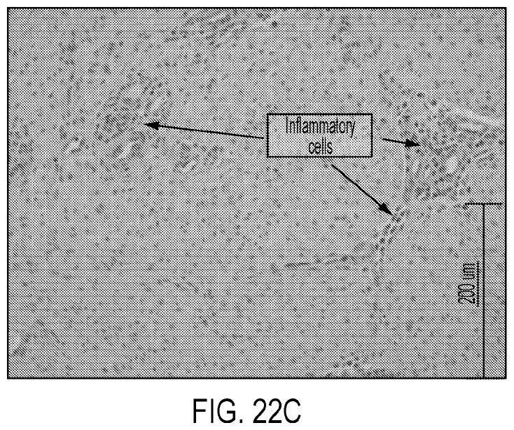

This Example demonstrates that, by histological analysis of tissues from porcine anal fistula tracts in Example 13, tissue remodeling leads to closure (healing) of the fistula tract.

Where treatment was withheld, the luminal surface of the fistula tract was lined with fibroblast-like cells, indicating that the luminal void would likely not close over time (FIG. 21(a)). At a higher magnification, inflammatory cells, fat cells, and immature blood vessels were observed (FIG. 21(b)). The image in FIG. 21(c) is identical to the position to the image in FIG. 21(a) but has been stained to highlight newly generated collagen fibres. All collagen features are shown in red. In places where the untreated fistula tract was closed, highly inflammatory cells and very few blood vessels were detected (FIG. 22(b) and FIG. 22(c)).

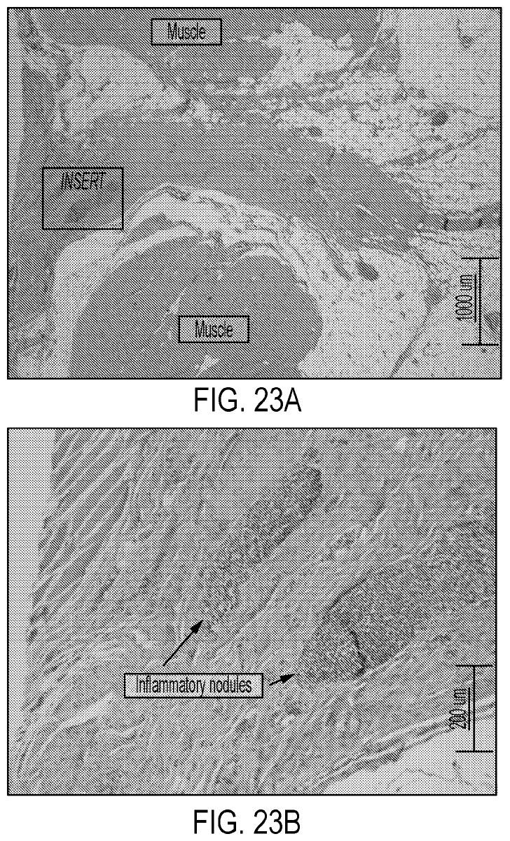

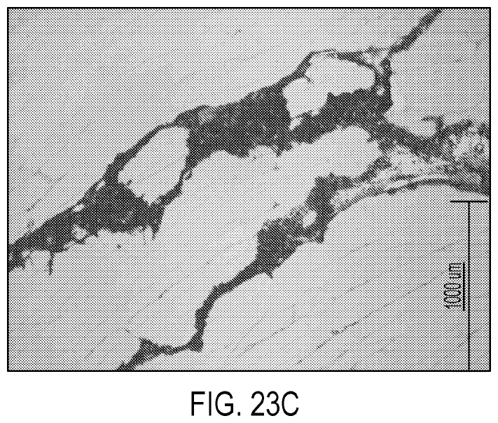

For treatment of the fistula tract with the Permacol scaffold alone, the fistula tract was observed in the muscle (FIG. 23(a)). At a higher magnification, one end of the fistula tract (closest to the external skin) shows two well-contained inflammatory nodules (FIG. 23(b)). The image in FIG. 23(c) is identical to the position to the image in FIG. 23(a) but has been stained to highlight newly generated collagen fibres.

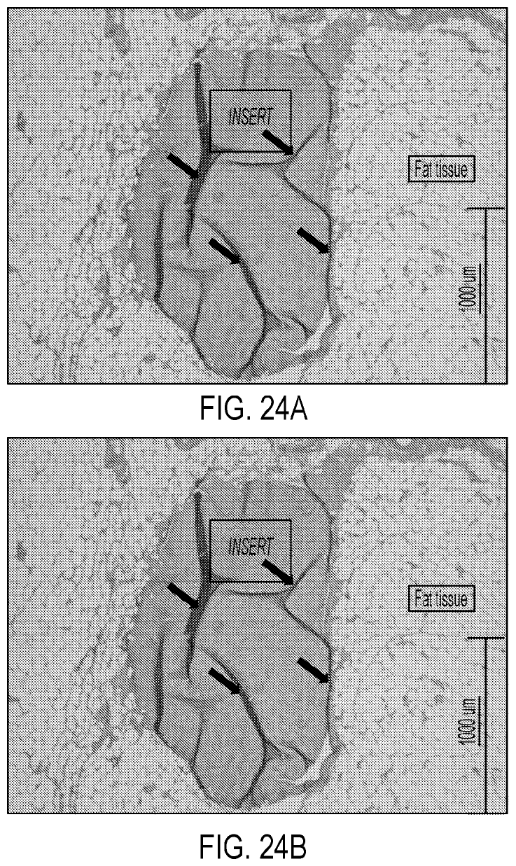

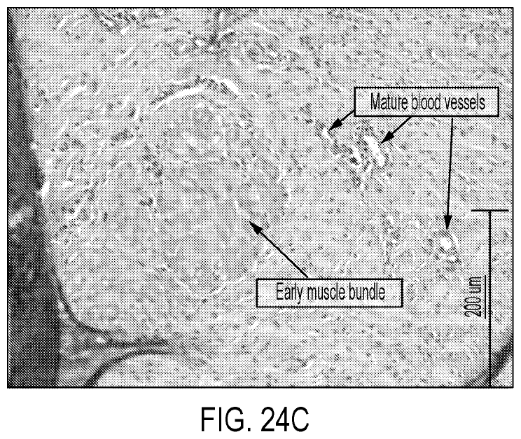

For treatment of the fistula tract with ATZ cells administrated with Permacol, the fistula tract within fat shows mostly fibrotic tissue with few inflammatory cells (FIG. 24(a)). The image in FIG. 24(b) is identical to the position to the image in FIG. 24(a) but has been stained to highlight newly generated collagen fibres throughout the infilled fistula tract.

(Note: The dark lines (represented by solid arrows) in FIG. 24 are folds within the tissue and are artefacts resulting from the sections not being stretched sufficiently prior to mounting on the slide.)

At a higher magnification of FIG. 24(a), the internal morphology of the ATZ and Permacol tract shows considerable regeneration with numerous mature blood vessels containing red blood cells at day 63 (FIG. 24(c)). Additionally, an apparent early muscle bundle with the different fibre types (i.e., possibly type I and type II) straining either light or darker shades of pink.

Taken together, this in vivo study provided histological evidence that collagen scaffold supports gender mismatched allogeneic adult porcine ATZ cells tissue remodeling for more effective closure (healing) of the fistula tract with mature blood vessels and early muscle bundle, supporting the multipotential capacity of ATZ cells.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A composition for treating an anal fistula, the composition comprising:
isolated anorectal transition zone (ATZ) stem cells and a pharmaceutically acceptable carrier.

2. The composition of claim 1, wherein the ATZ stem cells comprise progenitor cells.

3. The composition of claim 1, wherein the ATZ stem cells comprise multipotent stem cells.

4. The composition of any one of claims 1-3, wherein the ATZ stem cells can differentiate into endodermal cells, mesodermal cells, ectodermal cells, or a combination thereof.

5. The composition of any one of claims 1-4, wherein the ATZ stem cells are in vitro expanded ATZ (eATZ) cells.

6. The composition of any one of claims 1-5, wherein the ATZ stem cells express at least one marker selected from the group consisting of CD34, CD117, and CD184.

7. The composition of any one of claims 1-6, wherein the ATZ stem cells do not express detectable levels of CD45.

8. The composition of any one of claims 1-7, wherein the ATZ stem cells express at least one of NANOG and OCT4A.

9. The composition of any one of claims 1-8, wherein the cells are combined with an exogenous biocompatible scaffold.

10. The composition of claim 9, wherein the scaffold comprises a synthetic scaffold.

11. The composition of claim 10, wherein the scaffold comprises a biological scaffold.

12. The composition of claim 11, wherein the scaffold comprises collagen.

13. The composition of any one of claims 1-12, wherein the ATZ stem cells are porcine cells.

14. The composition of any one of claims 1-12, wherein the ATZ stem cells are human cells.

15. The composition of any one of claims 1-14 further comprising cryopreservation media.

16. The composition of any one of claims 1-15, wherein the composition is cryopreserved.

17. A pharmaceutical dosage form comprising the composition of any one of claims 1-16 disposed therein.

18. A method of preparing a pharmaceutical composition of any one of claims 1-15, the method comprising:
(a) obtaining ATZ tissue from a subject;
(b) enzymatically digesting the ATZ tissue with an enzyme to prepare a cell suspension;
(c) optionally combining at least a portion of the cell suspension with a cryopreservation media and cryopreserving the cell suspension; and
(d) combining the cell suspension of step (b) or optional step (c) with a pharmaceutically acceptable carrier.

19. The method of claim 18, wherein the cell suspension comprises ATZ stem cells.

20. The method of claim 19, wherein the ATZ stem cells comprise multipotent stem cells.

21. The method of claim 20, wherein the ATZ stem cells comprise progenitor cells.

22. The method of any one of claims 20-21, wherein the ATZ stem cells can differentiate into endodermal cells, mesodermal cells, ectodermal cells, or a combination thereof.

23. The method of any one of claims 21-22, wherein the ATZ stem cells express at least one marker selected from the group consisting of CD34, CD117 and CD184.

24. The method of any one of claims 20-23, wherein the ATZ stem cells do not express detectable levels of CD45.

25. The method of any one of claims 20-24, wherein the ATZ stem cells express NANOG and/or OCT4A.

26. The method of any one of claims 18-25, wherein the ATZ stem cells in the suspension are expanded in vitro.

27. The method of any one of claims 18-26, wherein, during step (d), cells in the cell suspension are combined with an exogenous biocompatible scaffold.

28. The method of claim 27, wherein the scaffold is a synthetic scaffold.

29. The method of claim 27, wherein the scaffold is a biological scaffold

30. The method of claim 27, wherein the scaffold comprises a collagen-based scaffold.

31. The method of any one of claims 18-30, wherein the subject is an anal fistula patient and the ATZ stem cells are autologous cells.

32. The method of any one of claims 18-30, wherein the subject is a donor and the ATZ stem cells are allogeneic to an anal fistula patient.

33. The method of any one of claims 18-32, wherein the subject is a human.

34. A pharmaceutical composition produced by the method of any one of claims 18-33.

35. A method of treating an anal fistula in a patient in need thereof, the method comprising: administering into the anal fistula an effective amount of the pharmaceutical composition of any one of claim 1-16 or 34, or the dosage form of claim 17, thereby to treat the anal fistula.

36. The method of claim 35, wherein the administration step causes the anal fistula to be closed or healed.

37. The method of claim 36 wherein the anal fistula is closed or healed by growth of fibrotic tissue.

38. The method of any one of claims 35-37, wherein the anal fistula is a perianal fistula.

39. The method of any one of claims 35-38, wherein the anal fistula is a refractory fistula.

40. The method of any one of claims 35-39, wherein the patient has Crohn's disease.

41. The method of any one of claims 35-39, where the subject has an anal fistula of unknown aetiology.

42. The composition of any one of claims 1-16 for use in the treatment of an anal fistula.
